(19) United States
(12) Patent Application Publication
(10) Pub. No.: US 2019/0162714 A1
(43) Pub. Date: May 30, 2019

(54) COGNITIVE CELL WITH CODED CHEMICALS FOR GENERATING OUTPUTS FROM ENVIRONMENTAL INPUTS AND METHOD OF USING SAME

(71) Applicant: Cemvita Technologies LLC, Houston, TX (US)

(72) Inventor: Tahereh KARIMI, Houston, TX (US)

(73) Assignee: Cemvita Technologies LLC, Houston, TX (US)

(21) Appl. No.: 16/312,820

(22) PCT Filed: Jul. 7, 2017
(86) PCT No.: PCT/US2017/041058

§ 371 Date: Dec. 21, 2018

Related U.S. Application Data
(60) Provisional application No. 62/359,339, filed on Jul. 7, 2016.
(60) Provisional application No. 62/361,037, filed on Jul. 12, 2016.

(51) Int. Cl. G01N 33/487; G06N 3/00; G16B 99/00; G16C 99/00; G01N 33/50

(52) U.S. Cl. CPC G01N 33/48771; G06N 3/002; G01N 33/50; G16C 99/00; G16B 99/00

(57) ABSTRACT

A synthetic, cognitive cell, system, and method for automatically generating an output based on an environmental input is disclosed. The cognitive cell includes an operator including chemical agents, and a coded chemical including polymers. Each of the polymers includes a sequence of affinity blocks of molecular groups arranged in predetermined patterns to define a multi-layered base code. Each of the affinity blocks includes a monomer with a sidechain, the sidechains having affinities to each other. At least a portion of the affinity blocks forming a gate switch defining a bridge between the environmental inputs and the chemical agent whereby, upon exposure to the environmental inputs, the gate switches trigger the chemical agent to perform an operation. The coded chemical and at least one chemical agent are contained within a natural or synthetic membrane.

### CROSS-REFERENCE TO RELATED APPLICATIONS

This application claims the benefit of U.S. Provisional Application No. 62/359,339, filed Jul. 7, 2016, and U.S. Provisional Application No. 62/361,037, filed Jul. 12, 2016, the entire contents of which are hereby incorporated by reference herein.

### BACKGROUND

The present disclosure relates generally to biotechnology, biochemistry, biophysics, chemistry, bioelectronics and computer sciences. More specifically, the present disclosure relates to the use of biologically inspired cells to perform various operations.

Computers were developed to perform algorithmic and logical functions, such as complex calculations. Computers have electrical switches corresponding to electrical inputs arranged in binary code. The inputs and corresponding code are programmed to receive and process the inputs according to defined algorithms, and to generate a desired output.

Computers have been used to monitor conditions and perform mechanical and electrical functions. Computers can be coupled to sensors to collect data from various sources, such as environmental conditions. In some cases, the sensors may be coupled to chemical matter to sense chemical parameters, such as composition, or biological matter to sense biological parameters. The data collected from the sensors may be stored in memories, and processed by the processing units. The processed data may be used to make decisions, and/or activate equipment to perform functions. Computers may be coupled to mechanical or electrical equipment to activate such equipment to operate according to programmed processes.

Despite advances in computer technology, there remains a need for devices capable of efficiently performing complex operations, such as autonomous functions. The present disclosure seeks to fill such needs.

### SUMMARY

In at least one aspect, the disclosure relates to a synthetic, cognitive cell for automatically generating an output based on an environmental input. The cognitive cell comprises an operator comprising chemical agents, and a coded chemical comprising polymers. Each of the polymers comprises a sequence of affinity blocks of molecular groups arranged in predetermined patterns to define a multi-layered base code. Each of the affinity blocks comprises a monomer with a sidechain, the sidechains having affinities to each other. At least a portion of the affinity blocks form a gate switch defining a bridge between the environmental inputs and the chemical agent whereby, upon exposure to the environmental inputs, the gate switches trigger the chemical agent to perform an operation. The coded chemical and at least one chemical agent are contained within a natural or synthetic membrane.

In at least one aspect, the disclosure relates to a synthetic, cognitive system for automatically generating an output based on an environmental input. The system includes at least one environmental input and synthetic cognitive cell. Each of the synthetic cognitive cell comprises an operator comprising chemical agents, and a coded chemical comprising polymers. Each of the polymers comprises a sequence of affinity blocks of molecular groups arranged in predetermined patterns to define a multi-layered base code. Each of the affinity blocks comprises a monomer with a sidechain, the sidechains having affinities to each other. At least a portion of the affinity blocks form a gate switch defining a bridge between the environmental inputs and the chemical agent whereby, upon exposure to the environmental inputs, the gate switches trigger the chemical agent to perform an operation. The coded chemical and at least one chemical agent are contained within a natural or synthetic membrane.

Finally, in another aspect, the disclosure relates to a method of making a synthetic cognitive cell for automatically generating an output based on an environmental input. The method comprises providing an operator comprising chemical agents; providing polymers comprising monomers; and forming a coded chemical. The forming involves selectively forming affinity blocks by modifying the monomers with sidechains (the sidechains having an affinity to each other), and arranging a sequence of the affinity blocks of the polymer into predetermined patterns of molecular groups defining a multi-layered base code and into gate switches defining a bridge between the environmental input and the chemical agent such that, on exposure to the environmental input, chemical reactions between the chemical agent and the coded chemical perform an operation. The method further comprises mixing the operator with the coded chemical to form a coded mixture, and applying the coded mixture to a membrane.

### BRIEF DESCRIPTION OF THE DRAWINGS

So that the above recited features and advantages of the present disclosure can be understood in detail, a more particular description of the invention, briefly summarized above, may be had by reference to the embodiments thereof that are illustrated in the appended drawings. The appended drawings illustrate example embodiments and are, therefore, not to be considered limiting of its scope. The figures are not necessarily to scale and certain features, and certain views of the figures may be shown exaggerated in scale or in schematic in the interest of clarity and conciseness.

FIG. 1 is a schematic diagram depicting a cognitive cell for performing various operations, the cognitive cell comprising a coded chemical and an operator.

FIGS. 2A-2C are schematic diagrams depicting example cognitive cells for performing energy, growth, and logic operations, respectively.

FIGS. 3A-3B show a schematic representative of layers and levels of coding and mechanisms for conditional de novo coding.

FIG. 4 is a schematic diagram depicting an example cognitive cell usable for performing the energy operation of FIG. 2A.

FIG. 5 is a schematic diagram depicting a plasmid with gate switches usable as material for the coded chemical.

FIG. 6A-6D are schematic diagrams depicting various proteins usable as the gate switches.

FIG. 7 is a schematic diagram depicting components of the operator.

FIGS. 8A and 8B are schematic diagrams depicting example energy operations.

FIG. 9 is a schematic diagram depicting an example growth operation.

FIG. 10 is a schematic diagram depicting chemical features of the growth operation.

FIG. 11 is a schematic diagram depicting the relationship between the gate switches for the growth operation.

FIG. 12 is a schematic diagram depicting an example traveling salesman problem (TSP) solvable by the logic operation.

FIG. 13 shows an exemplary Hybrid DNA/Protein algorithm, a bilayer coding system, for solving the TSP.

FIG. 14 shows a schematic representation of the coding sequences of the TSP.

### DETAILED DESCRIPTION

The description that follows includes exemplary apparatus, methods, techniques, and/or instruction sequences that embody techniques of the present subject matter. However, it is understood that the described embodiments may be practiced without these specific details.

### Introduction

The disclosure relates to cognitive techniques (e.g., cognitive cell, system and method) for generating an output based on an environmental input (e.g., sunlight, pH, temperature, etc.). A cognitive cell may be chemically coded to automatically respond to the environmental inputs and to trigger a chemical reaction that generates the desired output. The coded chemical (e.g., plasmid) may comprise affinity blocks arranged in predetermined patterns to define a base code comprising gate switches (e.g., clusters of molecules) capable of storing information in multiple layers to connected the coded chemicals to their environmental factors. These patterns may be pre-defined to store large volumes of information that are automatically triggered to provide an outcome upon receipt of a known environmental input in a multi-layered configuration that provides iterative processing across multiple directions to generate the predetermined, corresponding outcome, thereby reducing processing time.

The cognitive cell provides a cognitive chemistry platform with a molecular structure capable of embedding matrices of information in the chemicals. The information may be transduced by molecules using gate switches that act as molecular logic gates. The platform uses molecular machinery in the form of clusters of engineered coupled chemical reactions. The chemical reactions may be pre-engineered with recognition capacity over its environmental conditions. The cognitive cell may apply molecular sensors and coupling of environmental signals with internal logical operations of the cognitive cell using the logic gate switches.

The cognitive cells may be chemically and/or biologically programmed to correlate the coded chemicals in the cell to respond to the selected environmental inputs and cause reactions which generate the predetermined outputs. Such programming may employ single or multi-layered configurations of the gate switches capable of triggering the chemical agent to perform complex operations, such as generating energy (e.g., power), growing (e.g., regenerating, healing) cells, and/or performing logic (e.g., complex calculations), etc.

The cognitive techniques described herein seek to provide one or more of the following, among others: a renewable energy source, efficient processing, communication between cells and/or subjects, complex (e.g., multi-level) calculations, chemical and/or biological programming capabilities, natural and/or synthetic cell design, a commercially efficient autonomous polymer generator, a mechanism to convert energy (e.g., solar) directly to a desirable form (e.g., polymer or a desired structure with a highly specific geometrical architecture), and/or other capabilities based on needs of the system or in response to special environmental signals/conditions.

### Cognitive System

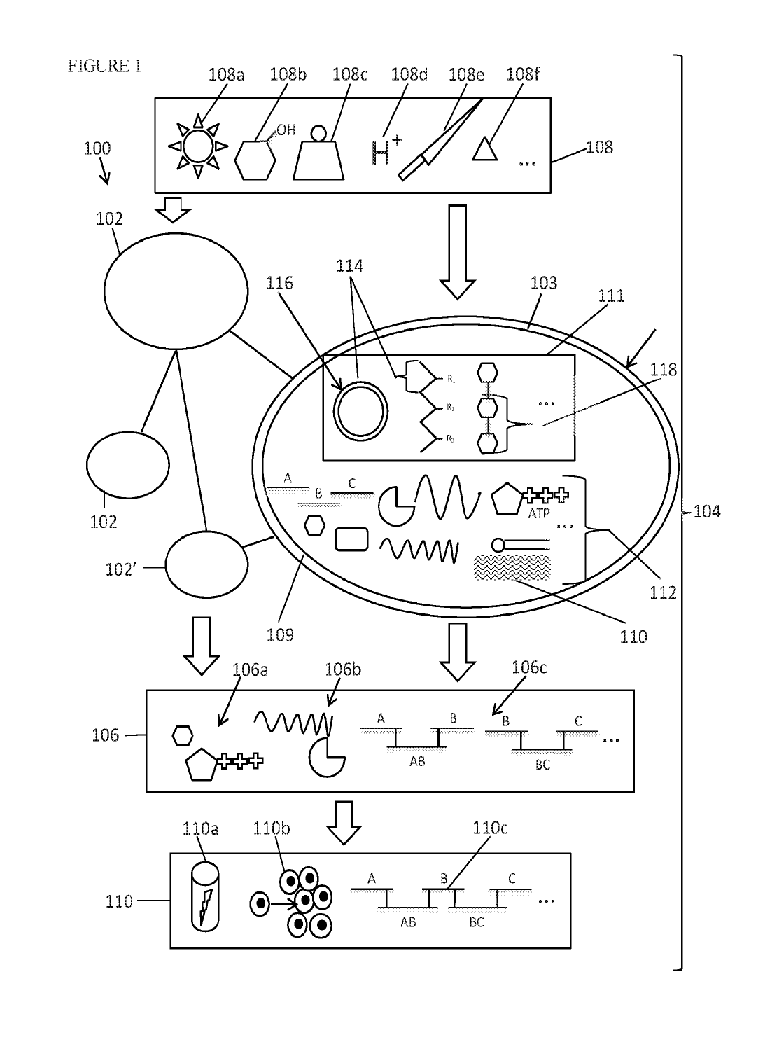

FIG. 1 is a schematic diagram depicting a cognitive system 100 that forms a chemical platform defining an autonomous system capable of performing various operations. This system 100 includes a cognitive cell 102 used to perform an operation 104, such as energy (e.g., self-fueling), growth (e.g., self-regenerating), logic (e.g., thinking, computing, decision making), and/or other operations. The cognitive cell 102 may be an operational unit in the form of molecular machinery made of a combination (or cluster) of chemicals capable of predetermined chemical reactions responsive to certain of the environmental inputs 108 and engineered to generate the desired outputs 106. The molecular machinery of the cognitive system operates based on interactions and affinities between molecules of the chemicals. The cognitive cell 102 may also be isolated, or part of a system or combination of cognitive cells 102 and/or non-cognitive cells 102′.

As shown by the operation 104 of FIG. 1, the cognitive cell 102 may be responsive to environmental inputs 108, such as light 108a, fuel 108b, pressure 108c, changes in pH as less limiting 108d, damage 108e, chemicals 108f, etc. While specific inputs are shown, any environmental input, factors, signal, etc. that is coupled with an internal operation in the cognitive cell 102 for making a specific output may be used. The cognitive cell 102 may be configured to receive the environmental inputs 108 and react in a manner that provides the desired output 106. As shown, the output 106 may be, for example, fuel 106a, cellular growth 106b, logic (e.g., calculations, scenarios) 106c, etc. The selected output 106 may then be used to provide one or more results 110, such as power 110a, regeneration 110b, a solution 110c, etc.

Part, or all of, the cognitive cell 102 may be natural or synthetic. The cognitive cell 102 may include a membrane 109 with a buffer 110 therein to support chemicals to perform the chemical reactions. The membrane 109 may be permeable, semipermeable, or impermeable depending on the desired application. For example, the membrane 109 may be made of cellulose to allow fluid flow therethrough. The buffer 110 may be a fluid (e.g., water phosphate buffer with a pH of about 7.4, etc.) housed in the membrane 109 to support the chemicals therein.

The cognitive cell 102 may include a variety of chemicals designed to respond to certain of the inputs 108 and generate the desired output 106. As shown in FIG. 1, the chemicals may include a combination of a coded chemical 111 responsive the environmental input 108 and an operator 112 to generate the output 106. One or more of the coded chemicals 111, operators 112, and/or other chemicals may be provided in one or more of the cognitive cells 102.

The coded chemicals 111 may be a natural or synthetic chemical or material with a predetermined molecular weight and physically observable mass usable for coding. The coded chemical 111, such as a polymer, and may comprise and/or be formed from, for example, deoxyribonucleic acid (DNA), graphene, aptamer, peptide, polysaccharide, protein, enzyme sensitive nanopolymer, synthetic nanopolymer, synthetic polysaccharide, polyethylene glycol, silica, and/or other chemicals.

The coded chemical 111 may comprise any programmable chemical and/or materials such as synthetic or/and natural polymers, made of a sequence of monomeric subunits, that are engineered or modified to store/embed codes (information) in chemical/electrostatic/hydrogen/polar and non-polar bonds between the affinity blocks in their molecular subunits, and/or in the pattern and sequence of their affinity blocks (e.g., as a sequence of alphabetic and digit characters in a language or coding system respectively). The coded chemicals may be engineered to be multifunctional (e.g., may be engineered to function as coding and energy-producing units at the same time), as well as being self-regulating in response to their environmental stimuli by direct connection to a wide range of logic gates which are connected to the environmental factors. For example, a logic gate may be responsive to a wide range of environmental stimuli including chemical, mechanical, physical, optical, electromagnetic, etc.

The coded chemical (or coded material) 111 may comprise monomers modified with sidechains to form affinity blocks 114. Examples of sidechains may include simple chemical groups such as hydroxyl, amino, carboxyl, carbonyl, sulfide, or they may include more complex groups such as natural or synthetic antigens and antibodies. The affinity blocks 114 may be arranged in predetermined patterns defining a base code 116. The affinity blocks 114 may be comprise of chemical groups in coding subunits of a coded chemical (material) that is engineered based on their affinity (e.g., through chemical, electrostatic, polar, hydrostatic interactions, etc.) with the other molecular groups in other coding sub-units and in some parts at least with a chemical agent to make a logic gate.

The molecules of the coded chemical 111 may be selected to act as storage units to provide means for storing information in an information storage matrix. The coded chemical 111 may store information in one or more layers of the molecules and/or portions of its chemical components. The affinity blocks 114 may be applied as chemical barcodes (e.g., representative of numbers/barcodes) for computation purposes in a cognitive chemistry system (e.g., real-time solving of NP problems).

Groups of the affinity blocks 114 may be arranged to define gate switches 118 within the base code 116. The gate switches 118 may be configured (i.e. programmed) to correspond to specific environmental inputs 108, and to the operator 112. The gate switches 118 may be optical (e.g., light sensitive switches) chemical (e.g., enzyme sensitive switch), physical (e.g., temperature sensitive switch), electromagnetic (e.g., UV sensitive switch), and/or other logic gates. The coded chemicals 111 may have recognition capacity over its environmental conditions which may enable the cognitive cell 102 to respond to certain environmental inputs 108 through the different affinity blocks 114 and logic gate switches 118 formed by the base code 116.

The configuration of the gate switches 118 may be used to define a bridge between the environmental inputs 108 and the operator 112. The logic gate switches 118 may function as linkers defining the bridge that connects the environmental inputs 108 to the to the internal operation system. For example, the gate switches may be a physical representation used to carry fundamental logical operations, such as NOT, AND, OR, NOR, XOR etc., used to determine when an environmental input 108 is present, which then may be used to trigger the operator 112 to generate its corresponding output 108. One or more of the gate switches 118 may be used to define logic gates (or circuits) used in performing a logic operation (e.g., Boolean function). The operation 104 may be triggered by existence of special environmental factors and/or sequential activation of the affinity blocks 114 and the logic gate switches 118.

The operator 112 may function as a module or a part of the molecular machinery of the cognitive cell 102 used for generating the output 106. The operator 112 may include one or more chemical agents, such as glucose, enzymes, proteins, RNA, lipids, ATP, vitamins, and/or other chemicals, responsive to certain of the gate switches 118. The chemical agents may be capable of generating part or all of the desire output 106 and/or operation 104 by performing a chemical reaction upon triggering by the gate switches 118.

The chemical agents may comprises a set of chemicals or molecules that are coupled with each other through chemical reactions and are functioning as modules of a molecular machinery to perform a desired operation/function initiating with one or multiple input(s). The chemical agents may be connected to other chemical agents internal to the cognitive cell(s), and may be connected to the environmental agents (factors) through the logic gates. Combinations of different chemical agents with various logic gates may provide a tight connection of the system to its environment and/or enable the system to indicate complex cognitive-like behaviors in response to various environmental inputs. Examples of chemical agents that may be used include adenosine diphosphate (ADP), ATP, guanosine diphosphate (GDP), GTP (guanosine triphosphate, nicotinamide adenine dinucleotide (NAD), Flavin adenine dinucleotide (FAD), transfer ribonucleic acid (tRNA), Ribulose 1,5 bisphosphate, Cobalaxime, tetracycline, rttA, nucleotides, ligase, amino acids, magnesium chloride, primers, glucose, and/or vitamins.

As also shown by FIG. 1, the cognitive system 100 may include multiple cognitive cells 102 to perform a complex operation and/or a chain of operations. One or more cognitive cells 102 may be merged, linked, stacked, and/or otherwise combined as desired. Each of the cognitive cells 102 may include their respective coded chemicals 111 and operators 112 that may be triggered by select environmental inputs 108 to generate select outputs 106. Optionally, the output of one cognitive cell 102 may act as the input to another cognitive cell. In this manner, the cognitive cells 102 may communicate directly or indirectly with inputs and/or other cognitive cells 102 to generate one or more outputs. Various combinations of inputs 108, cognitive cells, 102, and outputs 106 may be provided. The cognitive system 100 may optionally include non-cognitive cells 102′ that interact with the cognitive cells to facilitate and/or supplement operations performed by the cognitive cell 102.

While FIG. 1 shows a specific example of the cognitive cells 102, it will be appreciated that various combinations and/or variations of one or more cognitive cells, coded chemicals, operators, inputs, and/or other features may be provided and/or designed to achieve desired outputs. Additionally, while FIG. 1 shows the cognitive cells 102 used as the platform for performing the operation 104, it will be appreciated that the cognitive cells 102 may be used alone or in combination with computers and/or computing systems (e.g., processing systems with single layers of binary coding using electrical fields with bits) and/or other equipment (e.g., machinery) to perform one or more of the operations 104.

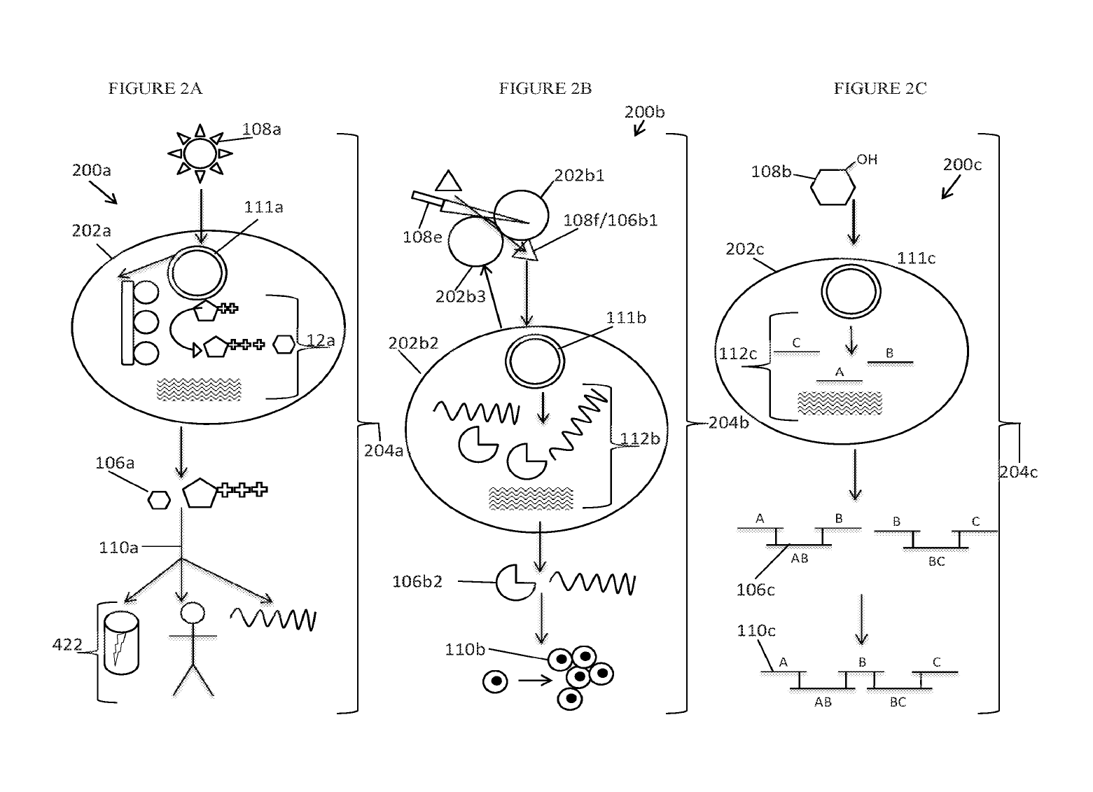

FIGS. 2A-2C depict examples of cognitive cells 202a, b1, b2, c and corresponding systems 200a,b,c for performing power, growth, and logic operations 204a-c, respectively. The cognitive cell 202a is a solar power cognitive cell capable of generating a power output 106a from a solar input 108a. The cognitive cells 202b1,b2 are part of a multi-stage, growth cognitive system capable of generating interconnected outputs 106b1,b2 from inputs 108e,f. The cognitive cell 202c is a logic cell capable of generating answers (routes) as output 106c from a fuel input 108b. As shown by these examples, the cognitive cell 202a may be programmed using the cognitive chemistry approach for providing autonomous energy, growth, and logic capabilities using cognitive coding.

### Cognitive Coding

The cognitive cells and systems may be configured as autonomous systems having features that provide cognitive, decision making, self-controlling, self-assembling, self-organizing, self-healing, self-replicating, self-fueling, and/or other capabilities. The cognitive system has capabilities relating to acquisition, perception, understanding, and remembering of information. The decision making may be independent of any outside mediator or operator. The self-controlling may relate to coding, controlling, and running its own processor, and may also have cognitive and decision making capabilities. The self-assembling may relate to assembling its own sub-units together and making a more complex structure or system based on thermodynamic laws (e.g., a self-assembling system would be generation of crystals of ice from H₂O at 0 C.° or polymerization of self-assembling polymers). The self-organizing system may relate to thermodynamic stability in structure, and provide integrated self-controlling (e.g., able to pass and transfer itself through different scales of complexity) and self-assembling features (e.g., the capability to generate its own sub-units and modules at different levels and scales of complexity). The self-fueling system may have the capability of producing and up-taking its own energy requirements from its environment without the need for any external mediator or operator. The autonomy may relate to independence from an external mediator or operator in the sense of coding, control, operation, structure formation, and assembling, as well as energy production. The self-healing may relate to regenerating its own damaged part by itself, independent of any external mediator or operator. The self-replicating may relate to replicating itself.

Design and generation of cognitive cells, systems, and methods may be based on cognitive chemistry which in addition to energy, information (code) is stored in the chemical bonds and/or electrostatic interactions between molecules. Cognitive chemistry comprises special class of multifunctional materials that are highly flexible in the sense of coding (information) storage capacity, as well as energy and mass (structure) production and/or transformation. Here, this feature of cognitive chemistry is defined as relativity of code, energy and mass versus relativity of energy and mass (in non-cognitive chemicals and materials).

The cognitive cells, systems, and methods may be used to feature one or the following: systems that are function based on the cognitive chemistry (relativity of code, energy and mass); design and creation of cognitive, decision making, self-assembling, self-controlling, self-organizing, self-healing, self-replicating, and/or self-fueling systems; design and creation of artificial intelligence, nano and microprocessors, Solving NP problems, Big data analysis, dynamic coding, conditional coding, multi-layer coding, production of renewable energies, solar cells, artificial photosynthesis, biofuels, bioreactors, agriculture and food industry, Synthetic life, symbiotic, integrated systems the embody both hardware and software (e.g., bioware), Cognitive diagnostic/therapeutic biomedical devices, Cognitive self-regenerating systems, Cognitive regeneration-stimulating biomedical products, Anti-aging treatment, Cognitive weight controlling biomedical devices, treatment of coding disorders in human/animals' diseases such as Cancer, Alzheimer, and genetic diseases, etc.

Design and generation of cognitive systems may be based on relativity of code, energy, and mass. The cognitive cells and/or systems may utilize “code” in industrial applications across disciplines including but not limited to energy and environment, biotech and life sciences, tech, agriculture, materials, medical, etc. For example, the cognitive cells and/or systems may be used in the field of biomedical science in designing and generation of cognitive biomedical devices that recognize the physiological and/or pathological conditions in human body. The cognitive cells and/or systems may be applied in treatment of diseases that currently there is no cure for them applying conventional therapeutic methods, such as cancer, HIV, auto immune diseases, Multiple Sclerosis, diabetes, and age related diseases (e.g., Alzheimer). Cognitive chemistry may involve creating systems that embody hardware and software (i.e., “bioware”), where even the smallest hardware building blocks show similar software capability as an overall unit in terms of intelligence. The bioware may have outputs about a first layer that acts as the input of the next layer, thereby providing a path-dependent process like a conditional set of dominoes.

A biologically inspired cognitive cells and systems may define the relativity of code, mass, and energy by virtue of a multi-layer, multi-stage, multi-level (multi-scale) biological coding system. As shown in Table 1 below, a biologically inspired cognitive system may have features comparable to computer systems, or employ features of computer systems, such as storage media, memory capacity, operators, coding subunits, operation, analysis/decision making capacity, hardware/software units, etc.

TABLE 1
COMPARISON OF CODING AND DATA PROCESSING
PROPERTIES OF HUMAN MADE COMPUTER SYSTEMS
VERSUS BIOLOGICAL SYSTEMS

| CODING | COMPUTER | |
|---|---|---|
| PROPERTIES | SYSTEMS | BIOLOGICAL SYSTEMS |
| Storage media | Monolayer storage | Multilayer storage media; |
| | media | e.g., Genes (DNA), |
| | (semiconductors) | Neurotransmitters |
| | | (proteins), Neural networks |
| | | (neural cells) |
| Memory capacity | High | Ultra-high through multiple |
| | | layers of coding systems and |
| | | multiple coding sub units, |
| | | DNA coding sub units: A, T, |
| | | C, G; Proteins' coding |
| | | subunits: 20 amino acid; |
| | | Biochemical protein/ |
| | | enzymatic signaling pathways |
| | | Neural networks: highly |
| | | dynamic growing in size and |
| | | complexity. |
| Operators | Logical (AND, OR, | Multilayer biochemical |
| | NOT, NOR, etc.) | operations |
| Coding subunits | 0, 1 | At the level of DNA: 4 |
| | | nucleotides |
| | | At the level of proteins: 20 |
| | | amino acids |
| | | At the level of signaling |
| | | pathways and neural net- |
| | | works: Proteins and enzyme |
| | | sub units, neural cell sub |
| | | units |
| Operation | Bitwise (sequential) | Simultaneous (Parallel) |
| | | Real time solution, as the |
| | | problem is defining, the |
| | | solution is forming by molec- |
| | | ular self-assembly and |
| | | interactions of affinity |
| | | blocks in coded chemicals |
| Analysis/Decision | No | Decision making capacity |
| Making Capacity | | |
| Hardware/Software | Separated hardware | Inseparable (integrated) |
| units | and software units | hardware and software |

In addition to computer capabilities, the cognitive cells and/or systems may be provided with layering usable for complex coding and/or storage. Table 2 shows examples of layered coding in a biological cognitive system.

TABLE 2
SEQUENTIAL LAYERS OF CODING AS WELL AS CODING
SUB-UNITS AT EACH LAYER IN BIOLOGICAL SYSTEMS

| LAYERS OF CODING | CODING SUB UNITS |
|---|---|
| DNA (genetic coding) | A, T, C, G |
| Epigenetic Coding (DNA methylation, | Methylation, Acetylation modifi- |
| Histone Acetylation) | cation sub units |
| Site specific DNA recognition | Zinc Fingers', TALENS' |
| sequences (Zinc Fingers, | Recognition domains |
| TALENs' coding system | |
| RNA Transcription and | Exons/Introns sub units |
| Alternative Splicing | |
| Amino acid coding system | 20 amino acids |
| Post Translational Modification | Glycosylation coding |
| Hormones | Peptides/Steroids |
| Protein signaling pathways | Protein and enzymes sub |
| | units of pathway |
| Proteome/metabolome signaling | Signaling pathways |
| net-works | |

The cognitive cells and/or systems may also be provided with scaled coding for passing information between various aspects of the biological system. Table 3 shows examples of scaled coding in a biological cognitive system.

TABLE 3
MULTI-SCALE CODING IN BIOLOGICAL SYSTEMS

| | CODING AND INFORMATION |
|---|---|
| SCALE OF CODING | PACKAGES |
| Nano (Micro) | Nucleic Acids, Amino Acids, |
| | Proteins, Saccharides, |
| Micro | Cells/Tissues |
| Macro (Systemic) | Chemical signal (e.g., Neuro |
| | transmitters Hormones, Abs, |
| | immune cells and blood circulation) |
| | Electrical signals (e.g., |
| | Electrical signals on neural |
| | system) |
| | Mechanical signals (e.g., |
| | Gravity sensing of cells, sensing |
| | of environmental modulus and |
| | elasticity by cells. |
| Information transformation | Hormones, signaling proteins, |
| between two organisms, | Neurotransmitters (e.g., |
| | Between mother and embryo) |
| Information transformation | Integration of information between |
| between generation, conserved | gametes (Sexual Replication) |
| coding and variety formation | |

The cognitive cells and/or systems may be provided with staged, dynamic, multi-stage coding that may occur according to various timing. Table 4 shows examples of stages in a biological cognitive system.

TABLE 4
EXAMPLES OF STAGES OF CODING
IN BIOLOGICAL SYSTEMS

| EXAMPLES OF DYNAMIC | |
|---|---|
| MULTI STAGE CODING | MECHANISMS |
| Stages development | 1-Stages of embryonic development |
| | including signaling pathways related |
| | to stem cells' fate determination, |
| | specification, differentiation, |
| | pattern formation during organogenesis |
| | and morphogenesis |
| | 2-Variety formation |
| | between species during the development |
| Aging | 1-Changes in expression pattern of |
| | age related genes including |
| | b-galactosidase, telomerase and |
| | telomere shortening and its sequential |
| | effect on the functionality of |
| | different types of cell in the body |
| | including stem cells |
| | 2-Apoptosis |
| Ag presentation and immunity | Changes in gene expression pattern of |
| formation | immune cells at different stages after |
| | ag presentation |
| Circadian cycles | Body internal clock |
| Dynamic responses to the | Fear and scape mechanisms and real time |
| environmental factors | changes in proteome/metabolome |
| Learning | Dynamic analysis and decision making |

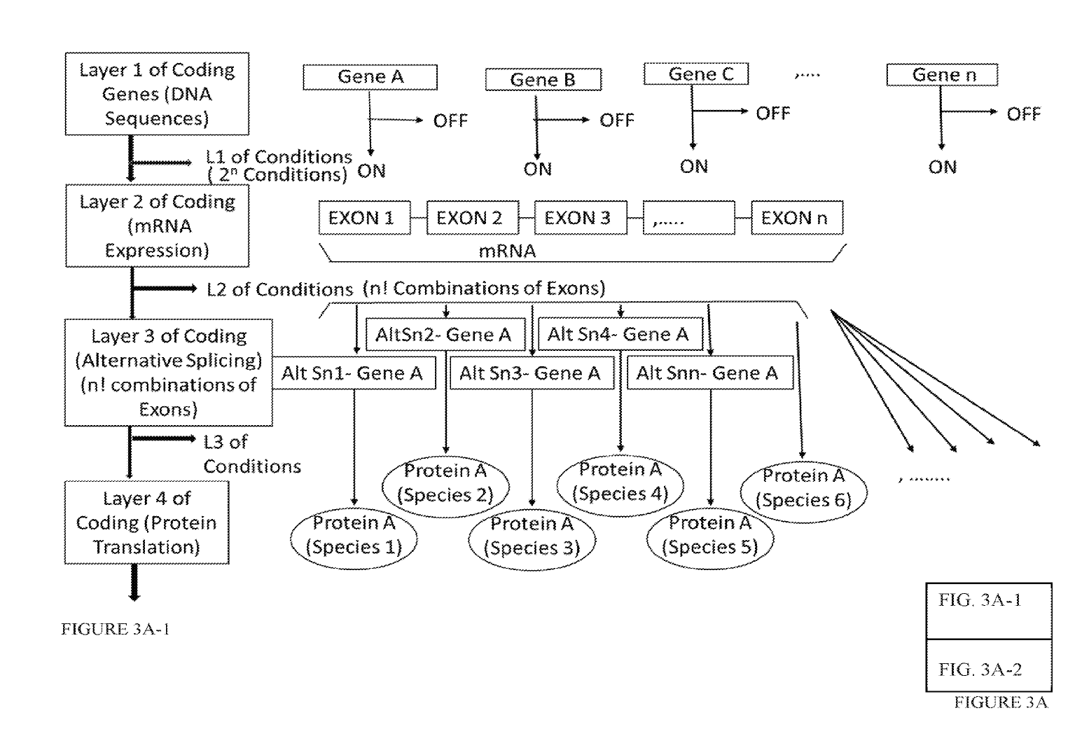

Each of the cognitive cells and/or components of the cognitive system may include various layers, scales and levels of coding and operations applied in a biologically inspired multi-layer, multi-scale, multi-stage/dynamic coding and data processing system. FIGS. 3A-3B show various layers and scales usable for coding in biological systems. FIG. 3A depicts layers 1-7 of dynamic conditional coding from genes A-n to proteins A-n. FIG. 3B depicts layers of dynamic conditional coding from formation of signaling path ways to the biological behaviors' outputs. These figures illustrate a summary of mechanisms of dynamic coding in various arrangements of coding sub-units at each layer of coding (which can be defined as alternative splicing and assembling) under the different environmental conditions. For example, at mRNA coding level, alternative splicing of exon in a gene with N exons, leads to N! (i.e., the product of integer from 1 to n) combinations of exons). Similar algorithms can be defined for other layers of coding.

Generation of the autonomous system, with capabilities of being cognitive, dynamic in coding and data processing and flexible in data processing and physical reactions to various environmental conditions and signals through dynamic structural changes, may involve a special form of real-time de novo coding instructed by environmental conditions (i.e., without the need of intermediates like coding by human for every single reaction to every single environmental condition). In addition, this system may be able to generate its own hardware in a flexible way based on the environmental conditions and signals (e.g., in the form of a physically and structurally real-time self-healing, self-replicating system).

The coding system may be classified in the sense of flexibility and capability to respond to the environmental conditions in categories, such as computer (conserved) coding and cognitive (de novo) coding. Computer coding may refer to current coding systems in silicon based computers in that an output of the system and responses to environmental conditions may be limited to the circumstances that are coded by a programmer, and the machine may not have the capability of self de novo coding based on new environmental conditions.

The cognitive coding may employ aspects of the computer coding, with the added capability of direct contact with the environment to be instructed and programmed directly by environmental condition and without the need of an intermediate programmer to code a predetermined response or boundary to an environmental condition. This may be used to expand the capability of current silicon based computers in response to the environmental conditions and signals to provide real time, self-de novo coding applying its basic coding sub-units. The expanded system may be provided with flexibility to be instructed by the environmental condition for generation of new codes for a particular reaction (output) to the environmental signals as the real-time input referred to as self de novo coding.

The cognitive coding may employ features of biological systems to provide the capability of real-time response to unlimited environmental conditions through de novo coding mechanisms applying basic coding sub-units at different layers and levels of coding. For example, an immune system provides real time de novo coding for synthesis of antibodies. Antibodies are synthesizing in the body just after the antigen presentation to the immune cells through the mechanism of alternative splicing for F-ab arm of Immunoglubulin (IgG) mRNA. After Ag presentation, a specific Ab based on the specific environmental condition (presentation of unlimited number of Ags in the nature) may be synthesized in the body. Therefore, even though a limited number of immunoglobulin G basic genes at the layer of DNA coding system (e.g., in human, mice, rats, etc.), immune system may have the capability of generation of various (unlimited) specific Abs against Ags that have not been in the body before. Just after their presentation to the body, immune system starts the process of de novo coding for the generation and synthesis of an Antibody against every single Ags presented to the body.

The cognitive processes and methods are coded as described herein for generation of a biologically inspired autonomous system by integration of the biologically inspired multilayer, multi-level dynamic cognitive decision maker system in the sense of coding, structure formation and energy utilization.

Multi-level directed self-assembly may be used in the formation of different structures with predefined morphology by applying the basic sub-units of coding and structure based on the environmental conditions through the mechanism of de novo coding. Designing such an autonomous system in coding, data processing and morphogenesis (flexibility in structure for example in a self-healing system) and independent energy production capability, may use a variety of features, such as coding features (e.g., coding media, layered coding), unity of software and hardware for directed self-assembly (e.g., for self-organization), flexibility, autonomy (e.g., self-energizing, self-deciding, self-coding, self-controlling, self-assembling, self-organizing and self-fueling), scaling (e.g., multi-layer, multi-level, multi-scale, conditional, dynamic, de novo coding), artificial photosynthesis, adaptability to environmental conditions, hardware regeneration, machinery for production or degradation of basic molecules into sub-units, and other features.

Various materials may be used in the cognitive system to provide a system capable of operation as a biologically inspired software capable of operating in a self-controlling state, while its hardware forms in a self-assembling, self-fueling, and self-operating state and in a self-organizing manner.

Different coded chemicals (materials) may be used for generation of a cognitive cell/system. Generation of cognitive cells/systems which indicating one or multiple features of autonomy (e.g., self-regeneration, self-fueling and self-regulation) requires the application of coded materials comprising sub-units with high flexibility in coding capacity, energy and mass (structure) production/transformation. For example, DNA, saccharides, proteins, graphene and aptamers are representative of materials that are highly flexible in coding, energy and mass production/transformation and are appropriate materials in generation of such autonomous systems.

For example, proteins are made of 20 amino acid sub units configurable in a large variety of combinations. Thousands of hundreds of proteins with large variety in structure and function can be generated through the assembling of amino acid coding sub units. The coding capability of the amino acids may be used for generation of a multilayer coding system, such as layers of amino acids and protein coding for solving problems, such as the NP or TSP problem of Example 3 (described herein). In addition, amino acids have the oxidation capability which may also be used to convert ATP into energy in biological systems. Therefore, amino acids and proteins can be defined as efficient materials with essential requirements of cognitive materials to be applied in generation of an autonomous system through the relativity in coding capability, mass (structure formation) and energy production/transformation.

Carbohydrates (e.g., saccharide) can be applied in generation of an autonomous system by providing the main requirements of the building blocks of an autonomous system through the high level of flexibility in the sense of coding capacity, energy and mass transformation. Carbohydrates may be applied in a cognitive cell/system for coding purpose. Glucose and other mono saccharides (e.g., fructose, galactore, mannose, ribose, etc.) have coding capacity by polymerization and providing specific sequences of glycosides. This may be done, for example, through post translational modification of proteins and formation of highly specific glycoproteins which may function as major mediators of signal transduction in biological systems. Glucose and other saccharides have physically defined structure and measurable molecular mass. Glucose and other saccharides can be oxidized and produce energy. For example in biological systems oxidation of one mole Glucose produces 38 mole ATP (which is defined as the energy currency in cells). See, e.g. Nelson D L, and Cox M M, Principles of Biochemistry, W.H. Free man and Company, New York, USA (2010, 202), the entire contents of which is hereby incorporated by reference herein.

Nucleic acids are another exemplary category of materials that can be applied in generation of an autonomous system by providing the building blocks of an autonomous system through the flexibility in the sense of coding capability, energy and mass (structure) transformation. Nucleic acid molecules (including ATP) are defined as a specialized coding chemical in biological systems. In addition, nucleic acids may be considered a specialized molecule for generation and transformation of energy. For example ATP functions as an energy currency in cells. Therefore, nucleic acids may provide a coding system with a measurable mass and the capability of formation of energy as well as various levels of structural complexities. ATP can be defined as an example of a material with relativity of code, energy and mass.

Structurally, nucleic acids, such as nucleotides and/or nucleosides, can be polymerized through phosphodiester bounds. As a result of polymerization reaction between nucleotides, different forms of nucleic acid polymers including DNA, mRNA, tRNA, rRNA with different levels of structural complexity and for different functional purposes may be formed. For instance, DNA is a specialized molecule for data storage, while mRNA is more specialized for data transportation and tRNA is specialized for translating and conversion of data from one layer of coding to the next one. However, rRNA almost specialized as a structural molecule in the formation of ribosome.

Unlike human made electrical binary coding system which is luck of features of mass for data, nucleic acids may be use for transformation from coding feature to mass. Nucleic acids may be used to provide a unique coding system with a measurable mass (e.g. MW of ATP=504.18 gr/mole) and the capability of formation of different structures. ATP can be defined as an example of materials with relativity in coding capability, energy production and mass formation (relativity in code, mass and energy).

Nucleic acids may be applied as coded chemicals in both forms of polymerized nucleotides (e.g. in the forms of DNA and mRNA) as well as single free nucleotides (such as cAMP as a signal transducer in a cognitive cell). DNA double helix structure may be used as a chemically and physically defined coding media. This structure may be used along with other coding systems, such as the binary coding system in current computers. In addition, novel coding and data processing algorithms may be defined for generation of multi-layer, multi stage dynamic coding system including multiple layers of DNA conserved coding layer, DNA epigenetic conditional and dynamic coding layer, mRNA alternative splicing de novo coding layer, for solving NP-hard problems'

Nucleic acids may also be used to produce energy, in the sense of relativity in energy. Nucleic acids can be oxidized and produce energy. ATP may be defined as an energy currency in cognitive cell.

Based on the multifunctional property of coded chemicals (e.g., ATP molecule), in the sense of coding capacity, energy and mass transformation; a novel equation of relativity may be defined as relativity of code, energy and mass. ATP may be used as a rechargeable molecular battery in a self-fueling solar cell and artificial photosynthesis system as in example 1 (described herein), code-based, self-fueling ATP battery with the capacity of production of energy and mass applying the relativity of code, energy and mass.

The biologically inspired multi-layer, multi-scale, de novo dynamic coding and data processing system may be used as CEMVITA™ Bioware, which is an integrated software and hardware system. CEMVITA™ Bioware may apply a multi-layer coding system, and DNA may function as the first layer of coding. Protein may function as the second layer of coding. In the first layer of coding which is DNA coding layer, 4 coding sub-units (quarters of code) including ATP, GTP, CTP and TTP (adenosine, guanosine, cytidine, and thymidine triphosphate, respectively) are used. CEMVITA™ algorithm may be used to explain the relativity f code, energy and mass for each quarter of ATP code. However similar calculations can be applied for other coding sub units at different layers of DNA, mRNA, protein and other coded chemical systems.

The equation of relativity of code energy and mass considers code as the third dimension of the physicochemical properties of a coded chemical and explains the transformation of code, energy and mass sub units to each other in a cognitive cell/system versus the equation of general relativity of energy, and mass E=mC² (Albert Einstein, 1995) which explains the transformation of energy and mass in a non-cognitive cell/system. The equation of relativity of code, energy, and mass explains and clarifies complex molecular features of a cognitive cell (including self-organization, self-regulation, self-fueling, self-regenerating and self-replication) that cannot be explain simply by currently defined laws of thermodynamics and/or general relativity of energy and mass.

Based on the General relativity law energy—mass and considering coding property of ATP molecule, as well as applying the molecular weight of ATP (MW: 507.18 gr/mole) and the equal amount of energy (J) stored in each ATP molecule, a novel equation for the relativity of code, energy and mass may be defined. ATP may be used as a rechargeable molecular battery in a cognitive cell.

DNA coding language is made of 4 sub-units of coding (quarters of code) including ATP, GTP, CTP, TTP. Here, ATP has been used for explaining the relativity of code, energy and mass in coded chemicals, but, similar calculations may be done for other coding sub-units at the layers of nucleic acids, amino acids, proteins, and any other coded chemicals which are flexible for energy and information storage in their chemical bonds or electrostatic interactions with other molecules

ATP is a highly specialized molecule of energy transportation and storage in biological systems including the capability for storage of light/solar energy in its chemical bounds (as chemical energy), and can be converted to the electrical, mechanical and other forms of energy. Light energy can be stored in ATP molecules through the natural or synthetic photosynthesis reactions in a cognitive cell/system.

The light used by for photosynthesis reaction in chloroplast has a wave length of about 700 nm. The energy (E) in a single photon is given by the plank equation at a wave length (λ) as follows:

$$\begin{matrix}E = hc \text{/} λ = ( 6.626 \times {10}^{- 3} J . S ) ( 3.00 \times {10}^{9} m \text{/} S ) ] \text{/} ( 7.00 \times {10}^{- 7} m ) \\ = 2.84 \times {10}^{9} J\end{matrix} \qquad Eq . ( 1 A )$$

An Einstein of light is Avogadro's number (6.022×10²³) of photons, the energy of one Einstein of photons at 700 nm is:

$$( 2.84 \times {10}^{- 19} J \text{/} photon ) ( 6.022 \times {10}^{23} photons \text{/} Einstein ) = 171.1 \times {10}^{4} J \text{/} Einstein \qquad Eq . ( 1 B )$$

Therefore, a mole of photons of red light has about five times the energy needed to produce a mole of ATP from ADP and Pi.

Based on the coding property of ATP as a nucleic acid sub unit and considering the molecular weight of ATP (MW=507.18 gr/mole), a new equation may be defined which explains the relativity of code, energy and mass in a cognitive (chemical) cell/system.

A Tara quarter of code (for a molecule of ATP) is defined as a coding unit that is relative in the sense of energy and mass. In other words, One (1) Tara quarter of code is defined as a unit of code as follows:
For energy: 4563.99×10¹³ J equivalent level of energy or 26.69×10¹³ Einstein photons
For mass: 507. 18 gr equivalent level of mass.
   Applying Eq. (1B), to these units, provides the following:
1 Tara quarter of code=26.69×10¹⁰ Einstein photon=4563.99×10¹³ J=507. 18 gr
1 Tara quarter code=MC2/(4563.94×10¹³)
1 Tara quarter code=(E/507.18×10⁻³)×C²

Using this approach, the cognitive system may use biological and/or chemical materials to achieve the desired coding. For example, using the carbohydrates, nucleic acids and amino acids may provide the flexibility in coding properties, energy production and mass formation, (relativity in code, energy and mass). In a cognitive chemistry system, main building blocks of structure, code, and energy are capable of transforming to each other based on the environmental conditions, energy levels and real time structural needs of the system.

In another example, other multifunctional synthetic chemicals such as the graphenes, aptamers, polyethylene glycol (PEG) and/or their modified forms in combination with carbohydrates, amino acids and nucleotides are other materials that can be applied as the appropriate candidate materials in the cognitive system. These and other materials may be combined to provide the cognitive system provide with integration of coding, energy and structural units together as well as designing and application of the materials with the relativity in coding properties, mass formation and energy production.

The coding of the cognitive system may be used for various applications, such as the solar application of Example 1, the growth application of Example 2, and/or the TSP application of Example 3.

### Example 1—Solar Power Cognitive Cell

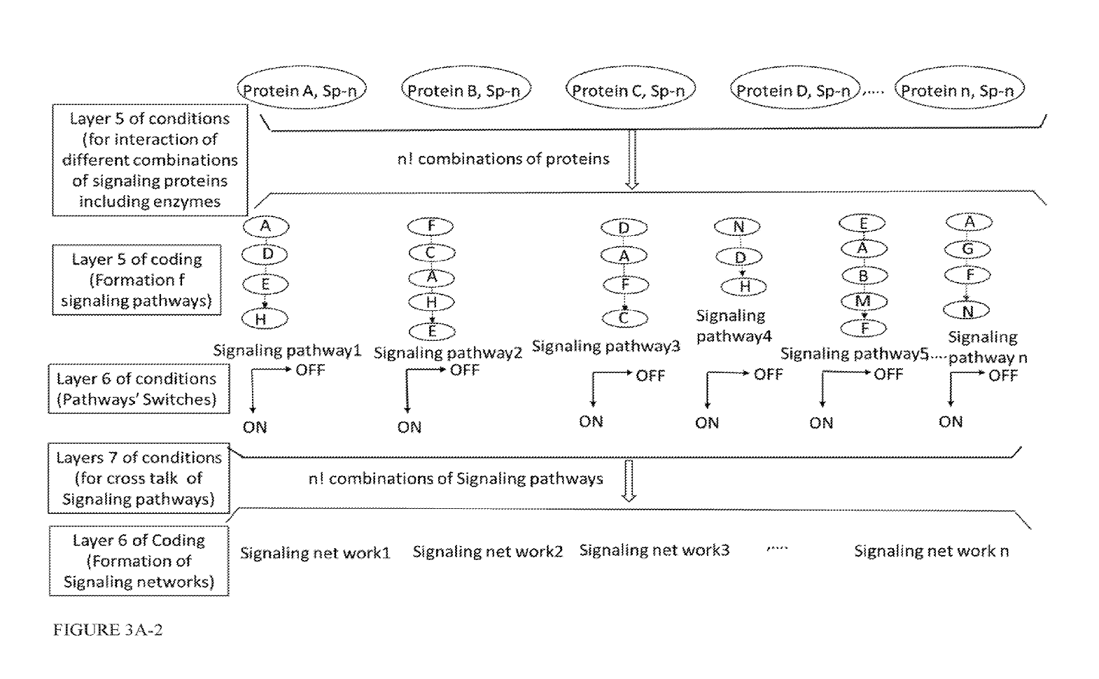

FIGS. 2A and 4 depict a solar power cognitive cell 202a including a plasmid coded chemical 111a reactive to the solar input 108a. Upon reaction to the solar input 108a, the coded chemical 111a triggers the operator 112a to generate the power output 106a. The power output 106a may be in the form of fuel which may be used to provide power 110A to a subject 422. The cognitive cell 202a may operate, for example, as an ATP-sugar battery chargeable by sunlight to release chemicals, such as sugar, from the cognitive cell 202a and into the subject 422 (e.g., through its bloodstream). This battery may also be used with or as an alternative to glucose-serum injection systems for human energy. While this example depicts a battery used to provide chemicals to an animal subject 422, the battery may be used as a power source for various other devices, including inanimate objects, such as batteries, electrical devices, and/or conversion to polymers.

In the solar power example depicted in FIG. 4, the cognitive cell 202a is used as a biomedical device for providing energy to a subject 422. The solar power cognitive cell 202a may be in the form of or included in a skin patch positioned on a subject 422. In this position, the cognitive cell 202a communicates directly with the subject 422. The cognitive cell 202a is externally positioned on a surface of the subject 422 for exposure to the solar input 108a, and referring to FIG. 1, performs the operation 204a to provide fuel to the subject 422 by performing a chemical reaction and passing a chemical through the membrane 103 into the subject. The membrane 103 of the cognitive cell 202a maintains the buffer 110, the coded chemical 111a and the operator 112a therein. The chemicals from the cognitive cell 202a are released through the membrane 103 and into the subject 422.

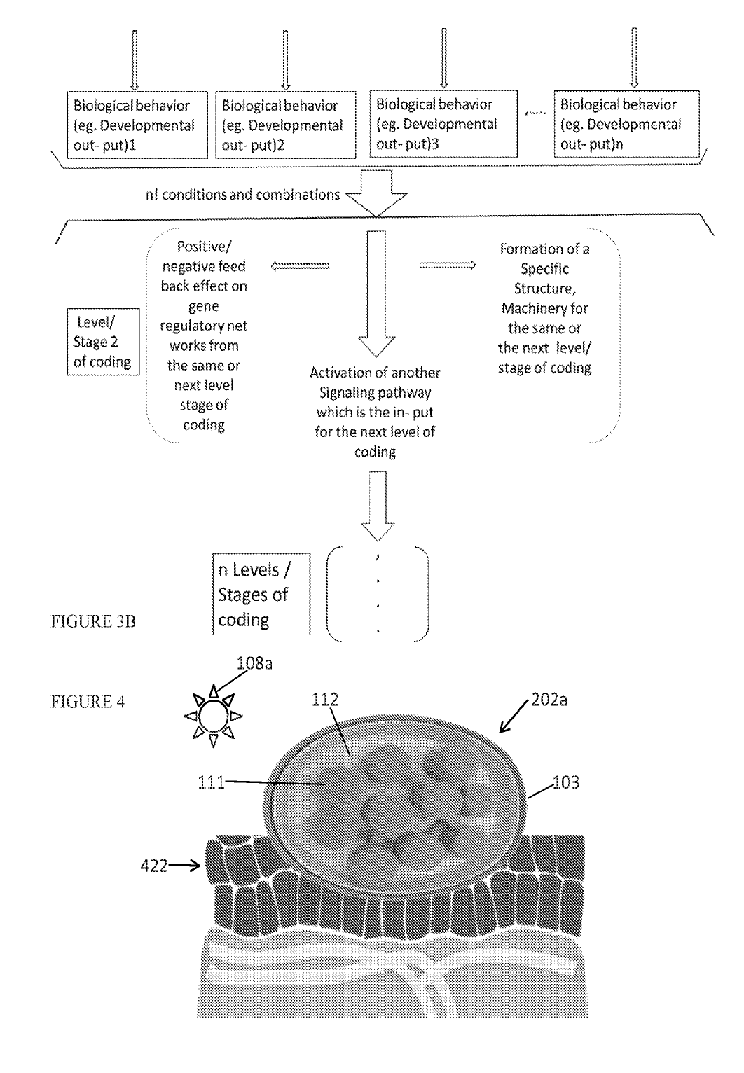

The coded chemical 111a may be a plasmid 516a made from nucleic acid as shown in FIG. 5. The coded chemicals 116 may also include amino acids arranged into proteins, which may be encoded by the plasmid 516. The coded chemicals 116 can be defined as representative of programmable materials in cognitive chemistry through their multi-functional chemical properties for energy and information storage/transformation in their own chemical bonds. In other examples, the coded chemical 111a may be coupled with chemical agents 112a flexible in the sense of information storage/transduction capacity as well as energy and mass transformation, such as graphenes, synthetic aptamers, hydrogels, different forms of nanopolymers and nanoparticles (programmable and degradable), etc.

Back bone expression of the plasmid PET-TALEN-HIS contains 6×His tag on C terminal of insert, and recombinant proteins of Spider silk, Insulin, Albumin, Phosphohexose lsomerase for cloning and expression in E.Col are used. In this example, the plasmid 516a is an expression plasmid PET TALEN-HIS including a Multiple Spidoin-I (MaSp1) gene, encoding Major ampullated Spidroin I protein (e.g., protein of Spider silk) of the Spider Nephila Clavipes. See, e.g., Xia et al., Native-sized recombinant spider silk protein produced in metabolically engineered Escherichia coli results in a strong fiber. Proc Natl Acad Sci. USA. 2010, 107:14059-63, the entire contents of which are hereby incorporated by reference herein.

The expression of the MaSp1 gene is linked to a gate switch 518a, triggered by an input of the tetracycline. The plasmid 516 also includes an insulin gene which is linked to a gate switch 518a with IPTG as an input, an Albumin gene under the control of a gate switch with Tamaxifen as an input, and a 4-Phosphohexose isomerase gene which produces an enzyme used as the operator 112a for conversion of fructose to glucose. The 4-phosphohexose isomerase gene expression is triggered by a gate switch which uses light as an input. The DNA sequences of the proteins of the operator 112a are attached to His Tag DNA Sequence, for the protein purification, and uses integration of specific DNA constructs of each gene from multiple cloning site of PET⋅TALEN⋅HIS (Add gene, Cat No. 40787).

FIG. 5 schematically shows a map of expression vectors in a polymer generator (nano-factory) system for the plasmid 516a. The plasmid 516a may have gate switches 518a formed from affinity blocks 514a made of nucleic acids of the plasmid 516a. The plasmid 516a may contain multiple gate switches 518a which are coupled to the expression of genes, which are in turn correlated to the operator 112a. The gate switches 518a may be coded to trigger the operator 112a to produce proteins for release from the cognitive cell 202a as outputs or as operators 112a to be used within the cognitive cell 202a.

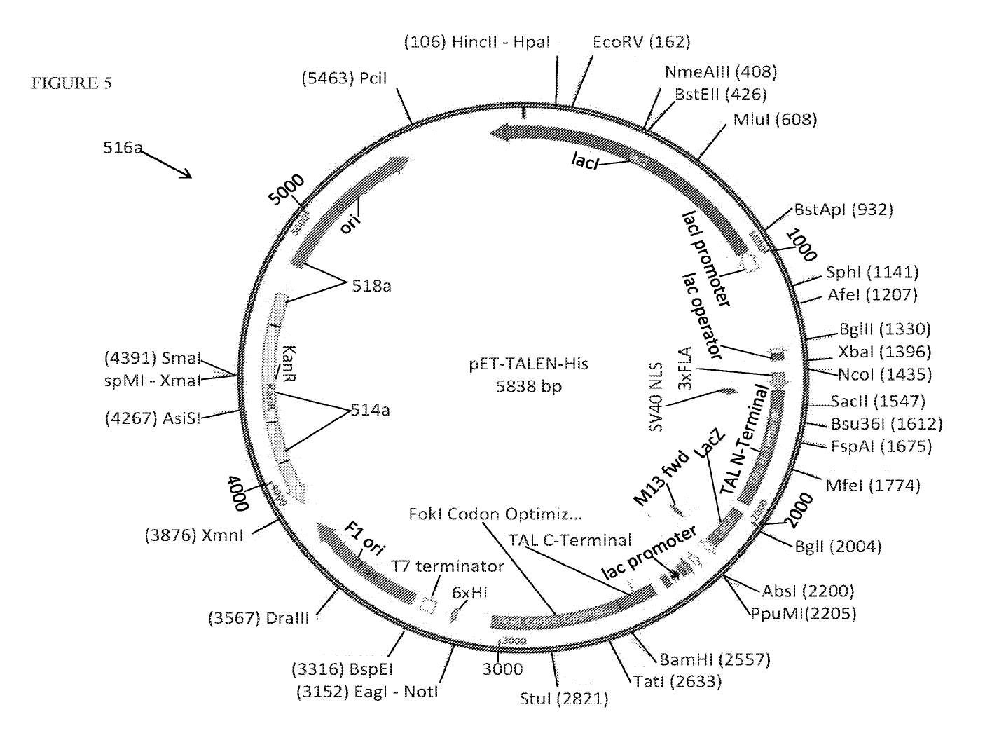

As shown in FIGS. 6A-6D, the gate switches 118 may be protein gate switches 618a-d which include combinations of amino acids (proteins) that define the activation/expression coding for the coded chemical 111a. These gate switches 618a-d consist of proteins that were encoded in a nucleic acid, for example the plasmid 516a of FIG. 5. FIGS. 6A-6D schematically illustrates four example gate switches 618a1-618a4 that may be formed by pairs of proteins 618a1,a2, b1,b2, c1,c2, d1,d2. The first protein 618a1, b1, c1, d1 and second protein 618a2,b2,c2,d2 work together to form a single gate switch 618a, b, c, d, respectively.

The gate switches 618a-d may be binary switches responsive to input 108a by either being in an on or off state. The ON or OFF state of the gate switch may then effect a change in the cognitive cell 202a, by, for example, acting as an input 108 to further gate switches, signaling the production and or activation of an operator 112a, and/or causing the cognitive cell 202a to produce the output 106a. For example, the gate switch 618a is an AND gate, which is in an ON state only when both the first protein 618a1 and the second protein 618a2 are activated by the input 108a. Gate switch 618b is an OR gate, which is in an ON state if either the first protein 618b1 or the second protein 618b2 is activated by the input 108a. Gate switch 618c is a NOR gate, which is in an ON state only if the first protein 618c1 is activated and the second protein 618c2 is not activated by the input. Gate switch 618d is a NOT gate, which is in an on state only if both the first protein 618d1 and the second protein 618d2 are not activated by the input.

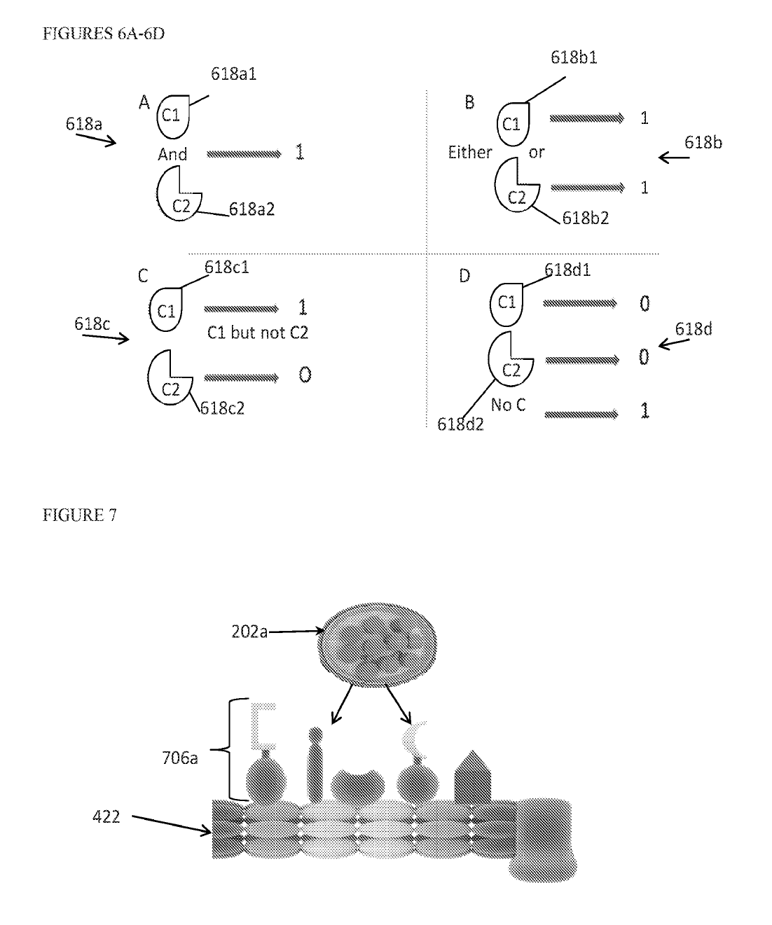

FIG. 7 shows example release of fuel 106a from the synthetic photosynthesis cognitive cell 202a. In this example, the operators 112b release an enzyme cluster 706a to the subject 422. The enzyme cluster 706a may include Ribulose-1,5 bisphosphate, carboxylase/oxygenase, phosphoglycerate kinase and glyceraldehyde-3-phosphatedehydrogenase, hexoisomerase, Aldolase, fructose 1,6 bisphosphatase, and phosphoglycerate kinase which may be immobilized on the artificial membrane 103 of cell 202a. In addition, the catalyst cobaxime may be bound to the membrane 103 to form a cobaxime coated membrane 706a.

The chemical reactions provided by the combination of the coded chemical 111a and the chemical agent 112a may provide both energy and information storage/transformation in molecules. These molecules may be usable as a programmable system to transform energy resources (e.g., solar energy inputs) into desired materials for a wide range of industrial/biomedical applications.

The output 106a generated by the reaction of the coded chemical 111a and the chemical agent 112a may generate chemical components as fuel 106a receivable by the subject 422. These chemicals may be selected for providing power to living organisms without toxic, explosive, flammable, or other damaging risks. Such chemicals may also be selected to operate in even extreme environmental conditions, such as aerospace condition for astronauts, space travelers, survival and rescue groups, etc. In this example, the output is ATP, but other chemicals, such as glucose, capable of generating energy may be used as the chemical output generated from the solar power cognitive cell. Energy production by ATP may not require the breaking of whole chemical bonds of the molecule. ATP may be used to produce energy by transferring of its high-energy phosphate groups when coupled with other chemical reactions. Each molecule of ATP may store about 12 kcal per mole (50 kJ mol⁻¹) from natural to artificial photosynthesis.

Chemical energy stored in the ATP molecule can be transferred to the biofuel molecules, or converted into the fuel. Considering that each ATP molecule is carrying three high energy phosphate groups and chemical energy in ATP molecule can be converted to the other forms of fuel usable as energy for various applications, such as electricity. ATP can also be applied as an efficient rechargeable power source. The cognitive cell 202a may provide an ATP-Glucose battery which may be integrated to an artificial photosynthesis system, such as a chemical solar cell.

Synthesis of the Cognitive Solar Cell

The chemical coding of the cognitive cell may employ features of photosynthesis present in green plants. Natural photosynthesis captures sunlight and converts it into chemical bonds of biomass in green plants. Through the photosynthetic processes and using solar energy, water splits into oxygen and hydrogen equivalents through the electron transfer chain in cytochrome systems of chloroplasts. The oxygen is released into the atmosphere where it is available for living organisms and maintenance of nature. The hydrogen equivalents are used to reduce CO₂ and for production of biomass in green plants.

During natural photosynthesis, light reactions in chloroplasts (including light absorption, water split and electron proton transfer) may provide the reducing equivalents in the form of reductive H₂. Nicotinamide adenine dinucleotide (NAD) is an enzymatic cofactor involved in redox reactions. NAD functions as an electron carrier, cycling between the oxidized (NAD) and reduced (NADH) forms. In the other words, NAD functions as a carrier for the released hydrogen from water splitting reaction. During natural photosynthesis, reduction of NAD to NADH occurs in cytochromes in chloroplast. The hydrogen is then used as a reducing equivalent for fixation of CO₂ and production of glucose.

Artificial photosynthesis may be performed in vitro by production of synthetic cytochromes. At least some artificial photosynthesis may involve complexity of cytochromes still and may result in lower efficiency than the natural photosynthetic systems. Techniques using photosynthesis are described in Huntley et al., CO₂ Mitigation And Renewable Oil From Photosynthetic Microbes: A New Appraisal, Mitigat. Adapt. Strat. Global Change, 12:573-608 (2007); Li Yet al., Biofuels from microalgae, Biotechnol Prog, 24:815-20 (2008); Atsumi et al., Direct Photosynthetic Recycling of Carbon Dioxide To Isobutyraldehyde, Nat Biotechnol, 27:1177-80 (2009); Niederholtmeyer et al., Engineering Cyanobacteria to Synthesize And Export Hydrophilic Products, Appl Environ Microbiol, 2010, 76:3462-6; Zhu et al., (2014); Kim D. et al., Artificial photosynthesis for sustainable fuel and chemical production, Angew, Chem. Int. Ed. 54:2-10 (2015); and McConnell I, Li G and Brudvig G W; Kim et al., Photochemical Production of NADH Using Cobaloxime Catalysts and Visible-Light Energy, Article: Inorganic Chemistry, American Chemical Society (2012); Kim et al., Visible-Light-Driven Photo production of Hydrogen Using Rhodium Catalysts and Platinum Nanoparticles with Formate, Article: American Chemical Society, pp. 25844-52 (2014); Park et al., Phototactic Guidance of a Tissue-Engineered Soft-Robotic Ray, Reports: Science Mag., pp. 158-62, Jul. 8, 2016; Han et al., Synergistic Effect of Pyrroloquinoline Quinone And Graphene Nano-Interface For Facile Fabrication Of Sensitive NADH Biosensor; Biosens Bioelectron, 89:422-429 (2017); Wang et al., Optimization of a Photo-regeneration system for NADH using Pristine Tio as a catalyst; and J Mol Catal. 2017, the entire contents of which are hereby incorporated by reference herein.

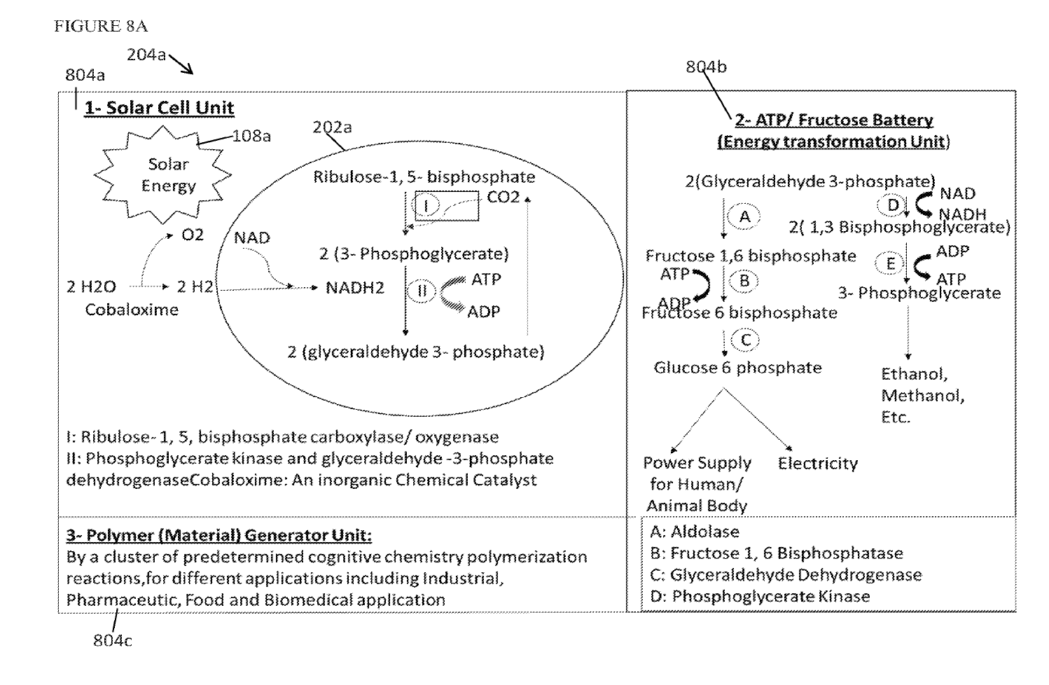

As shown by FIGS. 8A and 8B, the chemistry and processes used in the formation of the cognitive cell 202a may employ a synthetic form of photosynthesis using chemical coding. For example, the chemical coding may employ features of photosynthesis used in production of microbial biofuel cells, such as photosynthetic bacterial species and/or fuels or electricity for electronic devices/diesel engines. When applied to subjects, such as humans, the cognitive cell 202a may use a hybrid artificial photosynthesis/battery system applied as an energy source employing the combination of the chemical agent and the coded chemical.

Chemical materials, such as nucleic acids (e.g., ATP), may be used to form the coded chemicals and chemical agent to enable the cognitive cell to function as a high-energy rechargeable molecular battery. The cognitive cell 202a may be designed as a programmable ATP-battery that converts the solar energy input 108a to other forms of energy that are compatible for instance with electronic devices, a human body and/or other subjects 422. The solar power cognitive cell 202a may also be used to generate a hybrid artificial photosynthesis/ATP-sugar battery/and multi-functional polymer generator system that converts the solar energy input 108a to a wide range of materials, with different biochemical and biomechanical properties. The cognitive cell may be capable of transformation of solar energy to other renewable energy forms, thereby providing a self-generating polymer (material) generator.

The solar cognitive cell 202a may also be used as an alternate renewable energy source to supplement or replace fossil fuels and other non-renewable energy sources. The chemical structure of the cognitive cell may be used to store energy in the form of chemical bonds that may replicate photosynthesis in green plants. The solar cognitive cell 202a seeks to provide a synthetic source capable of providing one or more of the capacities of natural photosynthesis, such as oxygen production, solar energy up-take, energy storage, as well as synthesis of different types of materials by uptake of solar energy and CO₂ from atmosphere.

In an example, the cognitive cell 202a is synthesized in a configuration that produces a recombinant protein (e.g., spider silk, Insulin, albumin, collagen or each of the enzymes for the desired synthetic pathways such as different units of a solar cell, Glucose-ATP battery, etc.). Gene constructs of each recombinant protein/polymer are designed using the known DNA sequence relevant to each gene construct. The DNA constructs is synthesized by chemical methods, amplified by cloning in E. Coli, and subsequently purified. This is done by applying the Maxiprep method. See, e.g., ThermoFisher Scientific, Cat No, K210004. Each gene construct is inserted to a plasmid. The plasmid may be, for example, a PET TALEN-His plasmid (e.g., Addgene, Cat No. 40787). Each gene construct is inserted into back-bone plasmid through its multiple cloning site by applying the matching restriction enzyme for each gene construct. Expression plasmids containing genes of interest are cloned in E. Coli expression strain BL21 (DE3). E. Coli cells are cultured in 250 mL flasks containing 20 mL of LB medium in shaking incubator at 220 rpm. Cells are induced for 6 hours with the favorite inducible switches including Tetracycline, IPTG, Tamoxifen and light, etc.

When the OD 600 (optical density at 400 nm) reaches 0.4, samples are taken after 6 hours' induction for isolation of recombinant proteins and two-dimensional gel electrophoresis. Recombinant proteins are isolated and purified applying a His-Tag protein purification kit. The kit may be a plasma kit commercially available from www.clonetech.com (Clontech, Cat#635656). Purified proteins are sequenced and analyzed for physiological functionality. Matrix-Assisted Laser Desorption/Ionization on reflection Time-Of-Flight (MALDI-TOF) is used as the amino acid sequencing method.

The environment for formation of the cognitive cell 202a is defined to support the desired chemical reactions. The cognitive cell is formed in an in-vitro environment for production of recombinant proteins (Energy requirements provided by solar cell-ATP Battery). The in-vitro Environment for production of polymers uses a T7 mRNA synthesis kit, and an in vitro protein synthesis kit including free amino acids and expression of each protein under the control of its specific inducible switch. The enzymatic environmental reaction for production of each polymer conducted at pH 7.4, 37 C°, and energy of the system is provided by Solar Cell and Fructose, ATP battery. A microbial bioreactor environmental condition (Energy requirements provided by solar cell-ATP Battery) is used. Cloning of Plasmids into E. Coli for production of the enzymes for production of each polymer uses isolation and purification of Recombinant proteins from the bioreactor through His-affinity columns.

The coded chemical is formed by cell free protein production using In vitro RNA Transcription and Protein Translation. This translation is performed by coding DNA sequence related to each gene amplified by PCR (polymerase chain reaction) using specific primers. PCR products are purified and the quality of the generated DNA is determined. Using the in vitro transcription (IVT) process, mRNA is generated from the DNA product. Subsequently, the product is purified and treated with phosphatase to remove 5′-triphosphates. Sequentially, mRNA is purified and applied for in vitro translation. To this end, T-7 RNA Polymerase In-vitro Transcription kit (Thermofisher science) and In-vitro Translation kit (Thermo Scientific 1—step IVT kit are applied.

Analytical assays for measurement of NADH, Glyceraldehyde 3-phosphate, Fructose 6-Phosphate and ATP are performed. To evaluate the amount of NADH production, NADH fluorometric assay kit (APEx Bio) and NAD/NADH Quantification Kit (Sigma-Aldrich, Cat No. MHC037) are applied. Production levels of Glyceraldehyde 3-phosphate, Fructose-6 phosphate, and ATP, are evaluated applying Glyceraldehyde colorimetric assay kit (Sigma-Aldrich, Product No. 1.2.1.12), Fructose 6-phosphate assay kit (Sigma-Aldrich, Cat No. MAK020), and Luminescent ATP detection assay kit (Abcam, ab113849), respectively.

FIG. 8A shows the solar operation 204a involving synthetic photosynthesis depicted in compartments including the solar cell unit 804a, the ATP/Fructose Battery (Energy Transformation) Unit 804b, and the Polymer (Material) Generator Unit 804c. In the solar cell unit 804a, a cluster of synthetic chemical reactions are used for conversion of solar energy to the chemical energy by combination of an inorganic chemical catalyst (cobalaxime) and enzymatic reactions. The ATP/fructose battery unit 804b converts sugar based chemical energy to different forms of energy resources, such as sugars, ATP, biofuels, etc. The polymer generator unit 804c converts the solar energy into different polymers through polymerization reactions.

The enzyme clusters on artificial membranes (e.g., artificial cellulose membrane) are immobilized in the artificial photosynthesis-ATP/Glucose battery cognitive cell 202a. The solar input 108a causes a chemical reaction in the cognitive cell 202a which generates power 106a. The solar input 108a, together with carbon dioxide and water, reacts within the cognitive cell 202a. The coded chemical maps these inputs with the operators. The operators may include chemical agents in the form of clustered enzymes, such as ribulose-1,5, bisphosphate carboxylate/oxygenase, phosphoglycerate kinase, glyceraldehyde-3-phosphate dehydrogenase, ribulose 1,5-bisphosphate, aldolase, fructose 1,6 bisphosphatase, glyceraldehyde dehydrogenase, and phosphoglycerate kinase. In addition, the operator may include cobaloxime, which may be on an cobaloxime-coated cellulose-membrane with intermediate chemicals, such as NAD, ATP, and O₂.

The cognitive cell 202a uses synthetic photosynthesis reaction engineering by coupling chemical catalytic reactions with enzymatic reactions. Synthetic chemical pathways designed by coupling a chemical photocatalytic water splitting reaction with a cluster of synthetic enzymatic reaction to convert solar energy to chemical energy as rechargeable solar cell/battery compatible with human body. Inorganic catalysts, such as rhodium, platinum, TiO₂ nanoparticles, pyrroloquinoline quinone and graphenes nanoparticles, may be applied to facilitate the oxidoreductive chemical reactions. A catalyst is a substance that increases the rate of a chemical reaction without itself undergoing any permanent chemical change.

The solar operation depicted in solar unit 804a may be performed using an in vitro synthetic photosynthesis process may be used that applies a synthetic (inorganic) catalyst (e.g., cobaloxime) for splitting water molecules into oxygen and hydrogen. The photochemical water splitting reaction is coupled with the NAD reduction. The NADH functions as a reducing agent for the sequential CO₂ fixation reactions. Another synthetic chemical pathway is provided for CO₂ fixation. Ribulose 1, 5 bisphosphate (Sigma-Aldrich, Product No, R0878) is applied as the initial substrate for initiation of CO₂ fixation reactions. In the following chemical reactions, Ribulose 1, 5 bisphosphate is synthesized as a side product of the system for self-regeneration. Ribulose 1,5 bisphosphate carboxylase/oxidase converts Ribulose 1, 5 bisphosphate in two molecules of 3-phosphoglycerate. By addition of one molecule of CO₂, one molecule of NADH and one molecule of ATP to the next reaction results in the conversion of 3-phosphoglycerate into glyceraldehyde 3-phosphate which is 3-carbon sugar and converts to 6-carbon sugars sequentially as the final product of this pathway.

In this artificial photosynthesis process, molecules of water split in a synthetic photochemical reaction applying an inorganic (synthetic) catalyst (Cobaloxime). O₂ is released in to the atmosphere, and hydrogen is used for CO₂ fixation. Applying 2 sequential synthetic enzymatic reactions, the captured energy of sunlight which is stored in NADH₂ molecule is transferred to the glyceraldehyde 3-phosphate (a three-carbon sugar) (FIG. 2). Sequentially, glyceraldehyde-3 phosphate converts into Fructose and ATP.

The light 108a and water interact with cobaloxime to produce NADH. The NADH then interacts with the enzymes and the CO₂ to produce glucose. As shown in the ATP/Fructose battery compartment 804b, the enzymes may follow alternative pathways to fix the CO₂ into alternate metabolic products, such as ethanol and methanol. The synthetic chemical pathways may use an ATP/fructose battery transformation unit 804b to provide storage of power outputs, such as fructose (fruits' saccharide) molecules.

The power outputs may provide energy for human, electrical, and/or other needs for human/animal body use. Both fructose and 3-phosphoglycerate can be applied for production of different forms of biofuels including ethanol and methanol. Examples of the use of biofuels are described in Minteer et al., Enzyme-based Biofuel Cells, Curr Opin Biotechnol, 2007, 18:228-34; Zhu et al., Deep Oxidation Of Glucose In Enzymatic Fuel Cells Through A Synthetic Enzymatic Pathway Containing A Cascade Of Two Thermostable Dehydrogenases, Biosens Bioelectron. 2012, 36:110-5; and Sarris et al., Biotechnological Production Of Ethanol: Biochemistry, Process And Technologies, Engineering in Life Sciences. 2016, 16(4):307-329, the entire contents of which are hereby incorporated by reference herein.

Both Fructose and ATP are compatible with human/animals' body and can be applied as a power supply for human/animals' body, and for production of electricity in other applications. By conditional coding design and applying chemical switches, the cognitive cell 202a can be switched to be applied as a power supply for human body or as an electronic device.

FIG. 8B shows another schematic representation of a solar (synthetic photosynthesis) operation 804b using the solar cognitive cell 202a. The cognitive cell 202a is a hybrid solar cell, ATP/sugar battery and polymer generator units used in artificial photosynthesis. In this example, the synthetic photosynthesis cognitive cell 202a is shown between a pair of membranes 803a and 803b. The upper membrane 803a may be a synthetic membrane, such as a piece of glass. The lower membrane 803b may be a biological membrane, such as a lipid bilayer. The synthetic photosynthesis cognitive cell 202a includes several of the operators 812 immobilized together in the enzyme cluster and the cobalaxime coated membrane 803c. The operators 812 interact with the solar input 108a and air and water to produce glucose as an output 808b.

The lower membrane 803b includes glucose pumps, which allow the glucose 806a to pass out of the cognitive cell 202a, while other components of the cognitive cell 202a, such as the operators 812, remain between the membranes 803a,b. The glucose may then be used to produce a result 810a1-810a3. For example, the glucose 806a may be used to fuel a generator 809 to generate electricity 810a1. In another example, the cognitive cell 202a may be coupled to a subject 422 (as in FIG. 3) via nano-needles 830 to allow the glucose 806a to pass into the subjects 422 tissues to provide energy. The universal artificial photosynthesis system is insertable on skin of the subject 422 by nano needle. As schematically shown in FIG. 6, the artificial photosynthesis system matches with a human/animal body for insertion through to the skin to act as a power supply to provide energy to the body.

In a further example, depicted by FIG. 8B, the cognitive cell 202a may be used as an autonomous polymer generator by coupling the photosynthetic cognitive cell 202a with a polymer cognitive cell 802a. The glucose 806a may be used as an input to another cognitive cell, such as polymer generating cognitive cell 802a. In this case, the glucose 806a triggers the polymer generating cognitive cell 802a to produce a polymer 810a3, such as paper.

Applying a set of molecular switches in the chemicals, the cognitive cell 202a may be programmed for controlled production of various materials. For example, the polymer generator-system may be coupled with an electro-spinning or 3-D printing-system for generation of a wide range of materials with various chemical and mechanical properties and different industrial/biomedical applications. Expression plasmid PET-TALEN-HIS (Add gene, Cat No. 40787) 516 of FIG. 5 may be used for in vitro production of various recombinant proteins under the control of different chemical switches. For example, production of spider silk protein (MaSp-1) may controlled by tetracycline inducible switch; insulin production is controlled by IPTG switch; bovine milk Albumin under the control of Tamoxifen switch and phosphohexose isomerase under the control of a light-inducible switch.

As shown by the polymer generation unit 804c of FIG. 8A and the outputs 810a1-a3 of FIG. 8B, the cognitive cell 202a may be used to generate a variety of outputs, such as to convert solar energy (NAD) directly to a desirable polymer, and/or to provide self-generating synthetic and recyclable materials. This biologically inspired polymer generator system uses its own energy requirements for synthesis of polymers from sunlight. This polymer generator may be a system that has the production capacity for a wide range of synthetic recyclable materials, such as synthetic papers, recyclable polymers, recyclable bioplastic, etc., usable in various industrial applications and/or for pharmaceutics purposes (e.g., collagen, insulin, albumin, etc.). The process 204a-c may be used for generation of a variety of materials in various categories, such as synthetic paper (e.g., cellulose), fabrics (e.g., silk), composite materials for civil engineering applications (e.g., Spider silk-Chitosan composites), as well as nutrients (e.g., saccharides, starch, milk proteins), pharmaceutics products (insulin, collagen, etc.), etc.

### Example 2—Cognitive Cell Growth System

FIGS. 2A and 9-11 depict various aspects of the cognitive process 204b and the cognitive system 200b including the cognitive cells 202b1,b2 usable for growth (e.g., regeneration, self-healing, self-responsive, de novo synthesis of materials, etc.) of a subject 922. In this example, the cognitive cells 202b1,b2 generate a series of chemical reactions that allow the subject 922 to self-heal after damage. This example shows a cognitive chemistry soft-robotic system with different autonomous capacities including response to various types of environmental inputs, as well as growth capacities and real time responses to different types of the environmental inputs at macro, micro and molecular levels.

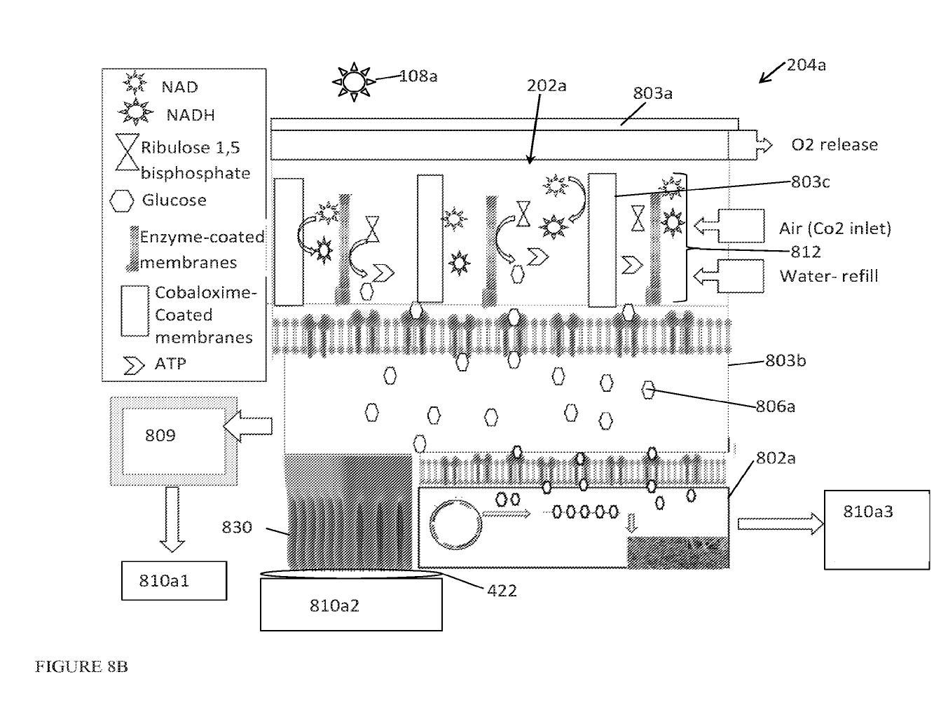

In the example of FIG. 9, the subject 922 is depicted as a starfish subject to damage. The cognitive system may be a self-regenerative symbiotic (synthetic life) system with the morphology of a starfish which possess similar properties of a living organism including, growth, self-regeneration, self-fueling, self-recognition and response to the environmental signals.

As an example, FIG. 9 demonstrates a self-regenerative-cognitive system with the morphology of a starfish subject to physical damage. The starfish 922 has a body with the solar cognitive cells 202a (e.g., ATP/glucose battery) centrally located therein, and arms 924 extending radially therefrom. The arms 924 have a collagen layer 926 with the cognitive cells 202b2 along an outer surface of the collagen layer 926, and the cognitive cells 202b1 positioned within the collagen layer 926. The collagen layer 926 isolates inner fluids 927a including the inner cells 202b2 from external fluids 927b including chemicals (e.g., enzymes) 928 outside the starfish 922.

The cognitive cells 202b1,b2 may be similar to the cognitive cell 202a of Example 1 with different chemistry to provide the series of the chemical reactions needed to generate the result 106b. The cognitive cells 202b1 may be, for example, fibroblasts comprising a coded chemical and an operator. The coded chemicals may comprise a plasmid (e.g., similar to the plasmid 516a of FIG. 5, but are carrying different codes for self-regeneration behavior of cognitive cells). The operator may comprise, for example, enzyme sensitive nano particles with tetracycline, or an inducible promoter such as Tetracycline Responsive Element (TRE).

The starfish 922 may have other cells, materials, chemicals, and/or other components, such as pacemaker cells 920 positioned about conductive nodes of the arms 924, purkinjie cells 932 in conductive fibers along the arms 924, and cardiomyocytes 934 positioned within the collagen layer 926.

Materials used in this autonomous system are arranged to provide both energy and information storage in their molecules, thereby providing tools for the generation of autonomous systems (self-responsive to the environmental signal, self-fueling and self-healing) that can transform energy resources (such as solar energy) into desired materials for its own regeneration after damages or producing different materials with a wide range of industrial/or biomedical application. For example, nucleic acids (in both monomeric and polymeric forms), amino acids, peptides, aptamers, carbon nanotubes and graphenes, and proteins are representative of programmable materials in cognitive chemistry by their multi-functional chemical properties for energy and information storage/transformation. Materials are not limited to nucleic acids and proteins, and may involve every synthetic material that is flexible in the sense of information storage/transformation capacity, as well as energy and mass transformation through their molecules.

The various cells and materials of the starfish 922 may be configured to perform one or more operations, such as power using the solar cognitive cell 202a as previously described and/or regeneration using the cognitive cells 202b1, b2. FIG. 9 shows various aspects of the self-fueling/self-healing process 204b for regeneration of the starfish 922 after damage. The process 204b is performed in stages, including stage W-initial, stage X-damage, stage Y-exposure, and stage Z-repair.

In the initial stage W, the synthetic photosynthesis cognitive cell 202a produces cellular energy (in the form of glucose) in response to sunlight (or other solar input) 108a. This glucose acts as a fuel input to power the starfish 922 and its other cognitive cells 202b1, b2 to carry out the self-healing operation 204b. The glucose may be generated using the solar process 202a as described, for example, with respect to FIGS. 2A and 8A-8B.

In damage stage X, when an arm 924 of the starfish 922 is damaged (e.g., cut) by an object (e.g., knife) which acts as an initial input 108e to the process 204b, the collagen layer 926 is opened and the layer of cognitive cells 202b2 are separated to create an opening in the arm 924 of the starfish 922. This opening allows the exterior fluid 927b to mix with the interior fluid 927a. This fluid 927b provides chemical 928 which acts as input 108b1.

In the reaction stage Y, the enzymes 928 in the exterior fluid are exposed to and react with the enzyme sensitive nanoparticles of the cognitive cell 202b1, causing the nanoparticles 912b1 to release the tetracycline 906b2. The output tetracycline 906b2 may then act as a chemical input 108b2 to the cognitive cells 202b2.

In growth stage Z, the tetracycline triggers the production of new collagen 926 and the cardiomyocytes 934. The new collagen 926′ rebuilds the structure of the arm 924 and the growth of new self-healing cells 202b2, sealing the damaged area of the starfish.

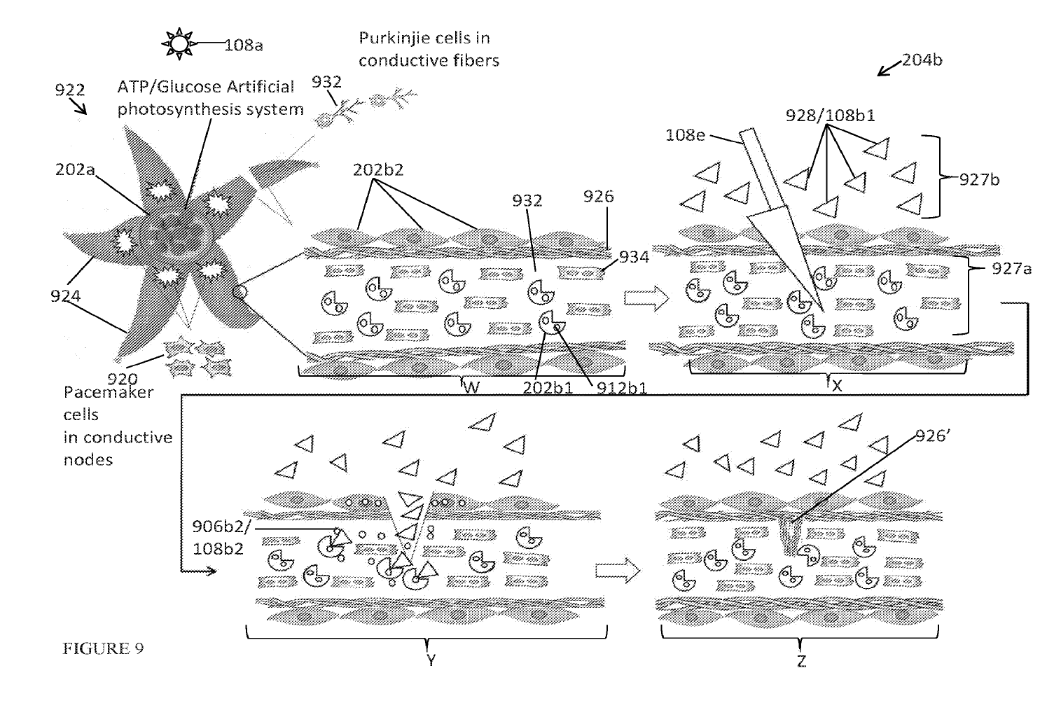

FIG. 10 shows the series of chemical reactions that occur during the cognitive process 204b. This figure provides a schematic representative of molecular mechanisms of conditional coding through inducible DNA switches. Various gene inducible logic gates and chemical logic gates in the nano-particles have been coupled together in engineering of autonomous properties of the system. As shown in these figures, the coded chemical 111b may include polymer 1011b1 formed from regeneration genes including TET responsive element (TRE) 1036a, FGF gene (FGF) 1036b, collagen gene (COL) 1036c and polymer 1011 b2 formed from primer genes including a CMV promotor 1036d, and rttA (TET transactivator gene) 1036e.

During stage W, the FGF 1036b and the COL 1036c are in the OFF state. In the damage stage X, the polymer 1011b1,b2 is exposed to the tetracycline 1012b1, and the polymer 1011b1,b2 is activated to combine rttA 1036e with the tetracycline 1012b1. This also releases cardiomyocytes.

In the reaction stage Y, the rttA 1036e/tetracycline 1012b1 attaches to the TRE 1036a which releases the growth cardiomyocyte 934. In the repair stage Z, the reactions of stage Y shift the FGF 1036b and the COL 1036c the ON position. In the ON position, the FGF 1036b causes proliferation of the fibroblasts and the COL 1036c causes production of collagen and regeneration of a damage matrix of the collagen along the opening of the arm 924.

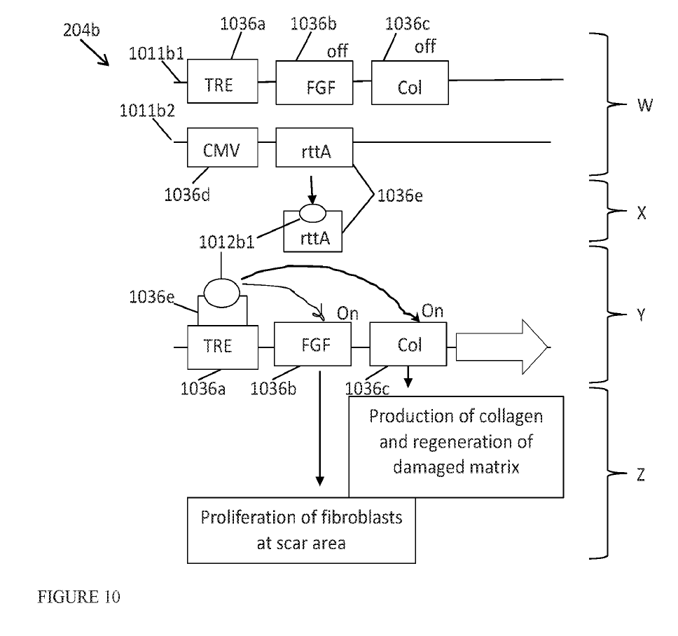

FIG. 11 illustrates various examples of protein coding modules that are functioning as logical gates in the cognitive chemistry system. Combinations of different logical gates are sufficient to process various kinds of logic information, and generate various outputs corresponding to certain inputs. As shown by FIG. 11, the gate switches 1118a-e may be arranged into logic circuits 1118a,b. The logic circuits 1118a,b act together to regulate the process 204b. In each logic circuit 1118a,b, each gate switch 1118a,b may generate an output that serves as an input to another gate switch 1118c,d, which may also provide another output that serves as another input to another gate switch 1118e. The out-put of each logic-gate can function as a positive or negative oscillator through their feed-back effects on the previous or next sets of logic gates. Combinations of logic gate switches and oscillators may be used to provide the self-regulation capacity for the system (i.e. multi-level coding).

In a manner similar to transistor switches in a computer, the gate switches 1118a-d in the logic circuits 1118a,b may act as positive or negative feedback to the logic circuit, in order to regulate the effect of the logic circuit. Each of the logic circuit 1118a,b includes a variety of the gate switches 1118a-e which provide a series of chemical reactions which generate outputs that serve as inputs to the next chemical reaction in the series.

FIG. 11 illustrates various examples of protein coding modules that are functioning as logical gates in the cognitive chemistry system. Combinations of different logical gates are sufficient to process various kinds of logic information. As shown in FIG. 11, the initial reaction of stage X occurs after damage (rupture) and exposure of the enzyme to the nanoparticles of the cognitive cell 202b1. The combination of the enzyme and nanoparticles shifts to the next gate switch 1118c in reaction stage Y to provide release of the tetracycline and induce the tetracycline switch 1118d to generate an output which acts as an input to switch 1118e. In stage Z, the outputs of switches 1118c,d may than act as an input to trigger switch 1118e to generate collagen or FGF which provides growth.

The logic gates in the cognitive chemistry system may be defined as different forms of chemical logic gate switches, such as nanoparticle logic gate switches, DNA promoter inducible switches, protein logic gate switches etc. The gate switches may be different combinations of cognitive-chemical gate-switches. Examples of nano-particle switches include enzyme-sensitive nanoparticle switch, temperature-sensitive nanoparticle switch, hydrolytic nanoparticle switches, pH-sensitive nanoparticle switches, UV/light-sensitive nanoparticle switches, and pressure-sensitive nanoparticles. Examples of DNA (promoter) inducible switches include TET inducible switch, IPTG/galactose inducible switch, estrogen/tamoxifen inducible switch, testosterone inducible switch, heat-shock inducible switch, and light inducible switch.

Each gate switch 1118a-e may act as a positive or negative feedback to either allow or deny generation of the outputs/inputs. Different combinations of cognitive-chemical gate-switches may be applied in the cognitive chemistry coding system with the capacity of performing more complex functions and responses to more complex environmental signals, through designing of multi-layer logical gate processes. Various forms of chemical switches may be applied in the cognitive chemistry computing system to carry the fundamental logical operations at different types of AND, NAND, NOT, OR, NOR, per the rules of Boolean logic.

The corresponding logical gate circuits may be used as a primary element for data processing. Because, as logic gate devices, they are able to adapt and learn. Therefore, various forms of protein logic gate devices can be applied as machine learning units in the cognitive coding system. In addition, different form of nanoparticles (that are programmed to be responsive to their environmental signals), also have been applied in designing the cognitive chemical logic gates.

The cognitive cells may be arranged and use to provide a special form of chemical based computing and data processing system made of cognitive materials to provide hardware and software integrated to each other in a cognitive chemistry system. The cognitive cells have recognition capacity of environmental signals applying various molecular sensors. The cells also provide capacity of both energy and information storage in their molecules (embedding of codes in materials).

The molecular modules and sub-units of the cognitive system are flexible in the sense of information storage/transduction through their chemical bonds as well as transformation of their structural geometry or energy production. The system has the flexibility for transformation of sub-units of coding, structure (mass) and energy to each other. Sub-units of the system are multi-functional and may be relative in the sense of coding capacity, energy and mass production and may shift from one function to another one, based on the environmental signals and for the highest level of flexibility in their autonomous behaviors.

Various environmental signaling factors can be coupled directly with different forms of the chemical logic gates. Direct coupling of an environmental signaling factors with an internal chemical logic gate, the system is empowered the system for direct and real-time response to the relevant environmental signaling factor/environmental condition. Different combinatorial layers of chemical logic gates/switches may be applied for more complex functions, in the cognitive chemistry coding system to empower the capacity of system for the real-time responses and consequently dynamic decision making based on the environmental signals.

Various chemical logic gates and switches may be engineered in various combinatorial forms of stimuli sensitive nanomaterials. Various stimuli sensitive nanomaterials may be applied separately in drug delivery researches. Stimuli sensitive nanomaterials may exhibit reversible or irreversible changes in their chemical structure or physical properties after special changes in environmental conditions such as magnetic fields, pH, ionic strength and enzyme activity. See, e.g., Hu et al., Enzyme Responsive Nomaterials for controlled Drug Delivery, Nanoscale, 6(21): 1227386, Nov. 7, 2014, the entire contents of which are hereby incorporated by reference herein. This approach may use applications of stimuli sensitive nanomaterials limited to very simple reactions based on one or two environmental signals, or more complex structures using various stimuli sensitive nanomaterials as building blocks and modules of logic gates.

Different combinations of stimuli sensitive nanomaterials can be applied as chemical logic gates in soft-robotic systems with the advantage of real-time response to the environmental signals. In addition, in complex logic gates and applying multiple layers of chemical logic gates the system can illustrate more complex cognitive behavior by real time response to multiple environmental signals. In addition, for more complex functions, applying the combinations of DNA coding language and conditional coding system (inducible promoter reporter-conditional expression system) the cognitive chemistry system can be programmed for real time reaction to the unlimited environmental conditions.

As shown by the process 204b as depicted in FIGS. 9-11, prior to damage, relevant enzymes of the nanoparticle exist in the sustaining culture media and are isolated from the nanoparticle by protection of the fibroblasts layer. The fibroblasts may be genetically programmed for production of collagen and FGF-1 under the control of a tetracycline switch (an inducible tetracycline promoter is inserted before the collagen and FGF-1 genes). In the presence of free tetracycline, the fibroblasts are stimulated to produce collagen and FGF-1. Collagen is a matrix protein and FGF-1 is a growth factor which causes the proliferation of fibroblasts. After a physical damage, and in the absence of the fibroblasts, the enzyme is exposed to the nano-particle and the tetracycline also releases. The tetracycline induces the production of collagen and FGF-1 in fibroblasts which causes the regeneration of damaged tissue.

The growth/self-healing example of FIGS. 9-11 provides another application for the cognitive chemical system and cognitive cells. The cognitive cells and systems may be used to design an autonomous decision maker system applying molecular based modules in cognitive chemistry computing systems. These systems may provide for information (code) storage in molecules, as well as energy and mass (material) production/transformation as relativity of code, energy and mass. This cognitive system acts as a chemistry computing system using a set of molecular modules responsive to input data much like the Boolean logic gates used in electronic based computation.

Synthesis of the Cognitive Growth Cell

The starfish 922 may be made by 3D printing of cell suspensions in layers including a layer of cardiomyocytes 934 covered by layers of fibroblasts at the bottom and top. Here, the morphology of system designed as a starfish. A pacemaker node at the central part of the body and pacemaker nodes at on top of each arm 924 are designed. In addition, electrical signals distributes in arms 924 through an array of purkinjie cells 932 at the middle of each arm 924. Pacemaker cells 830 are applied for induction of spontaneous depolarization and induction of contraction in the cardiomyocytes 934 and consequently spontaneous movement of the system.

The cognitive cells 202b1,b2 (e.g., fibroblasts and cardiomyocytes) may be formed by programming materials that respond to different environmental signals including light, chemical and mechanical signals applying. Various inducible promoters may be used for programming of fibroblast and cardiomyocytes for self-healing response to damage (e.g., injuries). Such programming may employ various plasmid techniques.

To program fibroblast and cardiomyocytes for responding to injuries as indicated in FIGS. 9-11, cells are transfected with a Lentiviral vector containing gene sequences of FGF-1, collagen and elastin under the control of a Tetracycline switch. Lentiviral vector system includes packing vector psPAX2 and envelope vector pMDS2.G (addgene). Tetracycline controlled transactivator (rtTA2) has been applied to function as an efficient biological genetic switch of the system. For this purpose Plasmid PNL-TREPiTT-EGFP delta U3-IRES2-EGFP is applied as a vector plasmid, and 293 FT cells are applied for viral packaging.

To program fibroblast and cardiomyocytes for responding to lights, cells are transfected with a Lentiviral vector containing GFP and a light inducible promoter-switch. Lentiviral vector system includes packing vector psPAX2 and envelope vector pMDS2.G (addgene). For this purpose, Plasmid PNL-TREPiTT-EGFP delta U3-IRES2-EGFP are applied as a vector plasmid, and 293 FT cells are applied for viral packaging.

To program the fibroblast for responding to the food (e.g., cellulose flavored with IPTG), cells are transfected with a Lentiviral vector containing cellulose gene and a biological IPTG switch. Lentiviral vector system includes packing vector psPAX2 and envelope vector pMDS2.G (addgene). For this purpose, Plasmid PNL-TREPiTT-EGFP delta U3-IRES2-EGFP (e.g., FIG. 5) is applied as a vector plasmid as a backbone, and 293 FT cells are applied for viral packaging.

To program the fibroblasts and the cardiomyocytes for photosynthesis and production and excretion of energy sources (e.g., glucose, amino acids and ATP), cells are transfected with a Lentiviral vector containing all genes of photosynthesis complex of purple bacteria under the control of a constitutive promoter CMV, applying a Lentiviral vector system including packing vector psPAX2 and envelope vector pMDS2.G (addgene). Plasmid PNL-REPiTT-EGFP delta U3-IRES2-EGFP is applied as a vector plasmid, 293 FT cells are applied for viral packaging.

Viral particle are produced in 293 T cells transfected with psPAX2, pMDS2. G vectors and plasmids containing every single gene of interest (above mentioned). Reprogramming of about 80% confluent ADSCs into proliferative state is accomplished by infection with the viral particles containing each gene of interest. Transfected cells are cultivated in 24 well cell culture plates by DMEM phenol red-free (Invitrogen) supplemented with 10% (vol/vol) Fetal bovine serum (FBS), 0.1 mM non-essential amino acids, 6 mM L-glutamate.

To further enhance the cell programming efficiency, this approach is combined with promoter-based Neomycine selection. Administration of Neomycine sequential to Lentiviral transfection, may leads to enrichment of genetically programed cell lineage populations for responding to each environmental signal.

The cardiomyocytes are provided by isolation and cultivation of neonatal rat cardiomyocytes. Freshly dissected whole rat neonatal hearts at day 1 to 3 are minced, incubated with Liberase DH (#05401054001) and Liberase™ (#05401119001) enzyme blends from Roche Diagnostics (Roche Blends) according to the instruction (Invitrogen). Minced heart tissues are incubated with Liberase DH or TM at the recommended concentration for about 35 minutes at about 37° C. The tissues are further disrupted to obtain a single cell suspension by pipetting up and down 25 to 30 times with a pipette fitted with a 1000 μL tip. Cells are washed twice with Hanks Balanced Salt Solution (HBSS).

After single cell suspensions are obtained from each method, total cell yield is determined using an Invitrogen™ Countess™ Automated Cell Counter and cell viability is determined by trypan blue exclusion assay. DMEM-F12 supplemented with 1% penicillin/streptomycin, 10% horse serum, NaHCO₃, BSA (bovine serum albumin), Sodium pyruvate, D-Glucose, Ascorbic acid, linoleic acid, transferrin, HEPES (4-92-hydroxyethyl)-1-piperazineethanesulonic acid), Sodium selenite). Total number of isolated cells is counted and cell viability estimated via trypan blue staining. Cells are seeded at a density of 125,000 viable cells per cm² on gelatin-coated multiwell-plates and placed in a humidified incubator at 37° C. and 5% CO₂. Cultures are left undisturbed for 24 hours and subsequent media changes are performed every 48 hours.

Pacemaker cells may be generated by isolation and cultivation of embryonic rat pacemaker cells. Undifferentiated Rat Embryonic stem cells (ESCs0) (provided from Lonza. Inc) are cultured in media containing 103 U/mL Leukemia Inhibitory Factor (LIF) (Chemicon) in 10 Cm² 0.1% gelatin coated dishes and passaged every 48 hours. Undifferentiated ESCs are transfected with promoter reporters for two specific pacemaker genes including GATA-6 and HCN-2. Proximal 1.5 Kb (−1.5 kb) region of the rat Gata-6 promoter/enhancer is inserted into the multiple cloning site of promoterless enhancer and red fluorescent protein (ERFP) vector containing hygromycin resistance gene (Clontech). The proximal 1.5 Kb (−1.5 kb region of the rat HCN-2 promoter/enhancer is inserted in to the multiple cloning site of promoterless enhancer and green fluorescent protein (EGFP) vector containing hygromycin resistance gene (Clontech). Undifferentiated ESCs are transfected with lentiviral virus particles containing both constructs as described in virus production and transduction methods earlier.

Once undifferentiated ESCs containing both vectors (promoter reporter GATA-6-ERFP and HCN-EGFP) have been produced, they start their differentiation, using the hanging-drop method. Briefly, 20 μL drop containing 200 ES cells in differentiation medium (growth medium without LIF) are placed on nontreated tissue culture petri dishes (Fiher) which is inverted for 3 days. The embryoid bodies (EBs) in hanging drop are suspended in differentiation medium in the same dishes for an additional 5 days.

At day 7 of differentiation, EBs are plated on to tissue culture dishes coated with 0.1% gelatin where they remained until used for next step detection and selection of conductive pacemaker cells (spontaneous depolarizing cells). On day 7 of differentiation, EBs are dispersed into single cells by incubating the EBs in trypsin for 5 minutes followed by mechanical dissociation using pipette.

After 5 minutes of centrifugation (1000 rpm), cells are suspended and plated into 0.1% gelatin coated dishes containing differentiation medium with 200 μg/mL G418 (Invitrogen). Each subsequent day cells are washed multiple times with Calcium magnesium. Free phosphate buffered saline and fresh medium containing 200 μg/ml G418 is added for a total 7 days. After 7 days of selection, cells are cultured for 4-6 days in medium containing no G418. After this time, cells are passaged using trypsin and are plated into 0.1% gelatin coated 35 mm dishes. Conductive cells expressing GATA-6 (RFP+) and HCN-2 (GFP+) cells are identified and sorted by flowcytometry method. mRNA expression and Immunocytochemistry (ICH) analysis for a panel of marker genes of pacemaker cells (including Tbx-3, Tbx-5, Tbx-18, Shox-2 and HCN 1, 2, 3, 4) are performed to confirm the differentiation of pacemaker cells.

Physiological functionality of pacemaker cells is determined through their hyperpolarization activated or funny current (If) which is specific ion current for spontaneously depolarizing pacemaker cells. Funny current (If) is elicited in the whole cell configuration by holding cells at −40 mv for 50 ms followed by 10 mV steps (2 s) to −130 mV and returned to −40 mV (50 ms) after each step. See, e.g., White S M, Claycomb W C, Embryonic stem cells form an organized, functional cardiac conduction system in vitro, Am J Physiol Heart Circ Physiol 288:H670-H679 (2005), the entire contents of which is hereby incorporated by reference herein.

In the next step, differentiated pace maker cells are applied in engineering of the spontaneous motile soft-robotic system in combination with hydrogel polymers. The main body of the cognitive chemistry autonomous system is made by a 3D printer applying the combinations of different types of cells suspended in a hydrogel (such as collagen or polyethylene glycol (PEG)).

### Example 3—Logic Cognitive Cell

FIGS. 2C and 12-17 depict aspects of the logic cognitive cell 202c and cognitive process 204c. In this example, the cognitive cell 202c is used as part of a computational cognitive system for solving a set of decision making problems in computer science using DNA/proteins for information storage in molecules. To solve the TSP (traveling sales person) problem, the cognitive cell 202c may be provided with a coded chemical 111c and an operator 112c capable of storing information concerning the TSP problem, and performing reactions to generate the solution. In this example, the coded chemical 111c may be a synthetic DNA, Proteins, synthetic polymers (e.g., PEG, Silica, etc.). The coded chemical may be modified with chemical sidechains including chemical groups such as NH₃, OH, COOH, SH, etc. The operator 112c may comprise, for example, free nucleotides, free amino acids, MgCl2, t RNA, primers, etc.

This logic operation 204c may be used to optimize efficiency of multi-node processes to solve problems, such as Nondeterministic Polynomial time (NP) problems. NP problems are a class of mathematical problems which have exponential complexity, for which an efficient solution may not be available. The NP complete problems are categorized as a class of decision problems in computational complexity theory. The travelling salesman problem (TSP) is an example of an NP-hard problem in combinatorial optimization, used in operations research and theoretical computer science. The TSP asks the following question: Given a list of cities and the distances between each pair of cities, what is the shortest possible route that visits each city exactly once and returns to the origin city? See, e.g., Garey et al., Computers and Intractability: A Guide to the theory of NP Completeness, W. H. Freeman & Company (1979) and Afaq et al., On the solutions to the traveling salesman problem using nature inspired computing techniques. IJCSI, 8(4); 21, 326-334 (2011), the entire contents of which is hereby incorporated by reference herein.

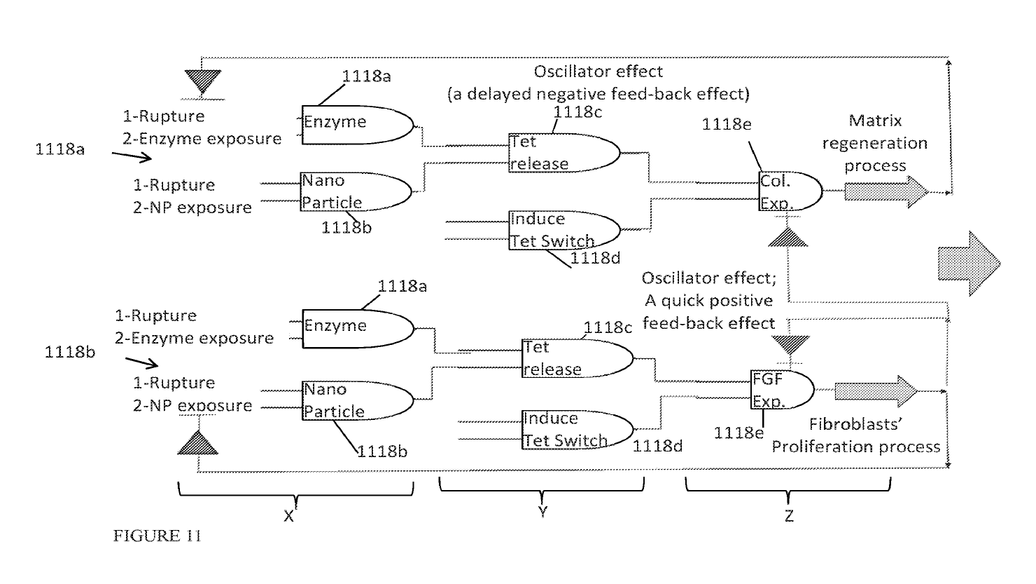

FIG. 12 schematically represents an example TSP 1200. The non-limiting example presented in FIG. 12 has 4 nodes (cities) 1230 connected by 12 routes (roads) 1232. The traveling salesman problem starts at a given city 1230, and travels along paths 1232 of various lengths such that each other city is visited exactly once before the route ends back at the starting city 1230. The paths start and end with city A. Cities are defined by A-D squares. Squares A1-D1 represent the initial 9 nucleotides of each city, squares A2-D2 represents the 9 end nucleotides of each city, and arrows represent the roads. The numbers on each arrow represents the cost on the given road.

The TSP finds a minimum cost (weight) path for a given set of cities (nodes or vertices) and roads (routes or edges). The path must pass each city exactly once and return to the original city. In a TSP with N cities, (N−1)! possible solutions exist. This means that for only 10 cities there are over 180 thousand combinations to try (since the start city is defined, there can be permutations on the remaining nine), resulting in 9!/2=181440 possible solutions.

One approach to solving this problem is to generate all possible routes, and then determine which possible route is shortest. To solve the TSP, a non-deterministic machine may be provided to explore each of the routes in parallel and computed in polynomial time, and to consider the possible routes, which may grow exponentially. Current computer operations may be used to process the possible routes sequentially where processing time is acceptable.

Where there are numerous different possible conditions for formation of networks, biological solutions such as, biologically inspired algorithms, genetic algorithms and DNA computing algorithms, may also be used for solving NP hard problems, especially when the number of nodes increases. See, e.g., Adleman L M, Molecular computation of solutions to combinatorial problems, Science, 266:1021-4 (1994), and Qian et al., Scaling up digital circuit computation with DNA strand displacement cascades, Science, 332:1196-201 (2011), the entire contents of which are hereby incorporated by reference herein.

Synthetic biological machinery in a logical cognitive cell (as in the logic cognitive cell 202c) may also be used to generate a set of possible solutions. The cognitive system may be used to solve NP hard problems using hundreds of thousands of biological signaling pathways in living cells by a few seconds per operation. The logic cognitive cell 202c define an optimal solution for the TSP by processing one or more of the routes simultaneously.

As shown by FIG. 12, the problem may have multiple nodes 1230 which define multiple routes 1232 resulting in a number of possible outcomes. The cognitive cell 202c may be coded (or programmed) to process the possible outcomes and determine an optimal solution. The structure of the cognitive cell 202c may provide a multi-layer system capable of storing large quantities of information, and processing a large number of possible outcomes 106c which may be used to define the solution 110c. The solution 110c may be defined to meet certain criteria, such as the shortest and/or least expensive route.

Biological solutions may be generated using techniques based on the physical properties of the DNA molecule including the length or melting temperature of DNA sequences, and/or using biological-inspired algorithms and/or techniques which may be limited to a single layer of coding and data processing and lack the adequate complexity of coding and data processing mechanisms in biological systems. Biological solutions may also be generated using the cognitive cells which use the DNA molecule for its coding properties through triplets of nucleotides to the amino acid codon system, which is involved in 20 codon sub units in a multi-layer, multi-stage dynamic biologically-inspired DNA/protein.

In cognitive systems, the multilayer DNA/protein may be encoded and molecular algorithms defined for solving NP problems, including those classified as hard NP problems. The TSP exemplified herein is given a graph consisting of nodes (vertices) linked by edges (binary paths) to find a route/path (concatenation of binary paths) which starts at a given node and ends at another given node, visiting every other node exactly once.

The optimal solution of data clustering can be defined through a NP hard problem such as shortest path. In a genetic pathway, nodes denote different genes, while roads are the correlation between the expression of two genes. The cognitive system may use a multi-layer hybrid DNA/protein algorithm for solving of TSP.

The biological coding and data processing in living systems involve multiple layers of coding, operation and machinery. Each layer of coding in a biological system may involve several coding sub-units (see, e.g., Table 1). Multiple layers of coding in biological systems may include coding languages of DNA, mRNA, amino acids, peptides, protein/enzymatic signaling path ways, systemic signal transduction through endocrine hormones and neurotransmitters and neural networks (see, e.g., Table 2). Biologically inspired computing algorithms with one layer of coding or different biological operations may be used for the coding. See, e.g., Hug H and Schuler R. Strategies for the development of a peptide computer. Bioinformatics. 2001; 17:364-8; Unger R and Moult J. Towards computing with proteins. Proteins. 2006; 63:53-64; De Castro L. N. Fundamentals of natural computing: an overview, Physics of life reviews, 2007, 4, 1-36; Roy S, Bioinspired Ant Algorithms, a review. I. J. Modern Education and computer science, 2013, 4, 25-35; Singh S., Lodhi E. A., Study of variation in TSP using genetic algorithm and its operator comparison, IJSCE, 2013, 3(2), 2231-2307; Chen Y J, Dalchau N, Srinivas N, Phillips A, Cardelli L, Soloveichik D and Seelig G. Programmable chemical controllers made from DNA. Nat Nanotechnol. 2013; 8:755-62; and Yang J, Dung R, Zhang Y, Cong M, Wang F, Tang G, An improved ant colony optimization (I-ACO) method for the quasi traveling salesman problem (Quasi-TSP), International Journal of Geographical information Science, 2015, 29(9), 1534-1551, the entire contents of which are hereby incorporated by reference herein.

Multi-layer biological coding and data processing in biological systems with different levels (scales) of complexity including nano (molecular), micro (or cell-cell interactions) as well as macro (or systematic) levels may be used (see, e.g., Table 3 and FIGS. 3A and 3B). Various layers of coding in biological systems with different nature and number of coding sub units may be used (see, e.g., Table 2). Data processing and operations in biological systems may occur through different layers of machinery by various biological motors. Examples of multi-layer machinery that may be used are provided in Tables 5 and 6 below:

TABLE 5
MULTI-LAYER MACHINERY (OPERATION)

| | BIOLOGICAL | IN-VITRO |
|---|---|---|
| LAYERS OF | MACHINERY/ | OPERATION |
| MACHINERY | OPERATION | MODELS |
| Genetic Layer | DNA Polymerase/Parallel | Polymerase chain |
| (DNA Replication | self-replication of | reaction (PCR) |
| Machinery) | system's data | |
| Genetic layer | CRISPER/CAS9 | (CRISPER/CAS genetic |
| (DNA Edition | Recombination System | engineering system |
| Machinery) | Transposes System | Homologous recombi- |
| | Restriction Enzymes | nation/Transposons' |
| | | gene editing system |
| Epigenetic | DNA Methylases/Histone | On Cheap Epigenetic |
| Machinery | acetyl transferases | data analysis |
| RNA | RNA Polymerases/Reverse | In vitro T7 Transcription |
| Transcription | Transcriptase | system/Expression |
| Machinery | | Plasmids |
| Protein synthesis | Ribosome/tRNA/ | In vitro cell free |
| Machinery | | translation system/ |
| | | Bacterial/Fungal/yeast |
| | | Translation systems |
| Biological net | ON/OFF pathway switches | e.g., In vitro induction |
| works' machinery | e.g., Kinases/phosphatases | of pluripotency/ |
| | | differentiation |
| | | pathways. |
| Cell-Cell | Gap Junction/Ion channels' | |
| communication | machinery | |

TABLE 6
MULTI SCALE MACHINERY

| SCALE OF MACHINERY | EXAMPLES |
|---|---|
| Molecular Machinery (Sub units | DNA Polymerase/RNA |
| of cells' machinery) | Polymerase/Restriction enzymes, |
| | different forms of enzymes. |
| Cell organelles' Machinery | Ribosome, Mitochondria, |
| (sub units of cells) | Chloroplast, Lysosome, . . . |
| Cells (sub units of tissues) | Various forms of Cells in |
| | different tissues |
| Tissue (sub units of organs | e.g., Nephron system in |
| and systems) | renal tissue. |
| Organs and Systems | Respiratory, Digestive, |
| | Cardiovascular, Neural, |
| | Hormonal, Reproductive |
| | machinery Systems |

The cognitive biological systems in data analysis and reactions to the environmental signals may be related to the multilayer nature of data processing in these systems (see, e.g., FIGS. 3A-3B). The multi-layer, multi-level biologically inspired coding and data processing algorithm may be used to solve even the most highly complex NP problems. Neural network which have been generated based on the simulation of data processing and operations in brain (or neural system) may be applied for solving NP problems, including TSP. See, e.g., Tarkov, M. S. Solving the traveling salesman problem using a recurrent neural network, American Analysis and Applications, 2015, 8 (3), 275-283, the entire contents of which is hereby incorporated by reference herein. Biologically inspired algorithms may be used in the coding and data processing through multiple layers and levels of complexity (nano, micro and macro). For example, gene regulatory net-works or protein-protein signal transduction net-works may be applied as a model for generation of biologically inspired data storage and processing systems.

The DNA may also be used as a coding molecule in biological system, as a conserved media for data storage during the natural development of various specious. Various biological systems at different levels of development (e.g., from prokaryotes to humans), may be models for the cognitive systems having the capability of responding to the environmental stimuli over time. At the molecular level, cognition may be based on the ability of proteins for data processing according to logical principles.

The level of structural flexibility of proteins and their interactional capability with each other and other biochemical components make them efficient switching elements of data processing networks in biological systems. For example, proteins play various roles in biological systems, including as enzymes, ion channels, receptors, membrane transporters and constructive elements of cells. In a protein-protein signal transduction and data processing net-work, proteins act as switching elements or signal transducers. See, e.g., Mark F, Klingmuller U, Decker K, (2009), Cellular signal processing, an information to the molecular mechanism of signal transduction. USA, Gaelan Science, Tylor and Francis Group; and Nelson D. L. and Cox M, (2012), Lehninger principles of biochemistry, 6th Edition, W.H. Freeman & Company, New York, USA, the entire contents of which are hereby incorporated by reference herein.

Switching capability of proteins is due to a conformational change induced by an in-put signal. Signal transduction can occur by a switching element protein due to the phosphorylation or interactions with cAMP or as a result of intermolecular Allosteric interaction between a regulatory domain (receiving the input signal) and a functional domain (transmitting the output signal). Signal transducing proteins are components of logical gates in biological data processing systems. Switches, can be used to carry out logical operations of the type NOT, AND, OR and NOR according to the rules of Boolean algebra. These operations may be used to process logical information.

The cognitive system may use capabilities of protein networks for data processing and dynamic coding based on environmental conditions, biologically inspired coding and data processing algorithms based on protein signal transduction systems, and/or global coding and operation systems having different levels of complexity including nano (molecular coding systems and operation), micro (cell-cell interaction networks) and macro (systemic signaling in humoral and neural system) in living organisms to provide a multilayer, multi-scale, multi stage biologically inspired algorithm with the capability of dynamic de novo coding based on environmental condition for solving NP problems. Different layers of coding may be defined to provide a higher data storage capacity. The coding system of each layer is completely different from the next layer (in the number and forms of sub units), however different layers of coding are highly interconnected to each other and in fact the out-put of each layer is the in-put for the next layer.

The cognitive system may be coded using to provide the problem solving capabilities. The cognitive chemical may use a biologically inspired algorithm which mimics the multi-layer, multi-stage, multi-level (multi-scale) coding systems in biological systems. See, e.g., Tables 1-5 above below which explain and classify different layers, scales and levels of coding and operations that have been applied in the biologically inspired multi-layer, multi-scale, multi-stage/dynamic coding and data processing system.

FIGS. 3A-3B show different layers and scales of coding in biological systems. In addition, FIGS. 3A-3B illustrates a brief summary of mechanisms of dynamic coding by various arrangements of coding sub-units at each layer of coding (which can be defined as alternative splicing and assembling) under the different environmental conditions. For example, at mRNA coding level, alternative splicing of exon in a gene with N exons, leads N! combinations of exons. Similar algorithms can be defined for other layers of coding. Conditional dynamic de novo coding system may be sufficient to generate all possible solutions of a TSP problem with N cities (which is defined as (n−1)!/2 conditions) even at mRNA level of coding. In addition, as the number of coding layers may increase the rate of data processing and the capability of system for data storage will be increased.

The coded chemical 111c of the cognitive cell 202c (FIG. 2C) may have logic gate and methods for a hybrid DNA/protein computing algorithm, usable as the multilayer biologically inspired data storage and processing algorithm for solving the traveling salesman problem (TSP). As an example the present application presents a hybrid DNA/protein coding system for solving TSP. However, this multi-layer coding system can be applied for solving TSP or other TSP problems by any other combinations of coding layers as defined herein. By increasing the number of coding layers the capacity of system for data storage and analysis/processing may be increased respectively.

FIGS. 3A and 3B schematically show how a coded chemical may be used to generate an extremely large number of secondary coding chemicals. FIG. 3A shows DNA as a coding chemical, separated into genes. Each gene may either be on or off. Through the process of transcription, the genes which are on are expressed as messenger ribonucleic acid (mRNA) exons which contains equivalent information to the DNA genes. The mRNA exons may undergo alternative splicing to create alternate mRNA exons such that a greater number of alternate mRNA exons exist than the original number of genes. The alternate mRNA exons are built from the information originally contained in the DNA genes, and each alternate mRNA exon no longer matches the information in each gene. Through the process of translation, the information in the alternate mRNA exons or the mRNA exons are encoded into a string of amino acids, which form a protein.

FIG. 3B continues by showing how the proteins may come together to form a signaling pathway comprising a set of the proteins. Because there may be as many signaling pathways as there are combinations of proteins, there are now many more signally pathways than there were DNA genes used as the original coding chemical. In this way, systems may be designed which use a relatively small number of DNA genes as coding chemical building blocks to create a very large number of possible signaling pathways, which may also be considered to be coding chemicals of the system.

In a manner similar to how the proteins come together to form signally pathways, the signaling pathways may come together to form signaling networks. As well as representing the information of the original DNA genes as a coding chemical, the signaling networks may also act as gate switches (see, e.g., the gate switches 118 of FIG. 1), facilitators 112, or even inputs 108 or outputs 106. Thus, the information in the coding chemicals (such as the DNA genes), may exist at multiple levels throughout the cognitive system as shown by Table 7 below:

TABLE 7
MULTI-LAYER GENE REGULATION

| | Examples of | |
|---|---|---|
| | Regulators/Switches | In vitro models |
| Levels of | systems at different | of Gene regulatory |
| regulation | layers of Coding | switch systems |
| DNA | Non coding dna sequences | Inducible switches |
| | including promoters, | (tet, light, galactose |
| | gene enhancer and activators, | inducible switches) |
| | modulators, tet response | |
| | element, steroid response | |
| | element | |
| RNA | Non protein coding rnas such | In vitro guide rna |
| | as shrna, | gene regulation |
| | irna, linc rna, guide rna | system |
| Protein | Transcription Factors, | Transcription factors |
| Small | Steroids such as dexamethasone, | Different forms of small |
| molecules | testosterones ant etc. | molecules |

Amino Acids may also be clustered based on the PKA of their respective side chains.

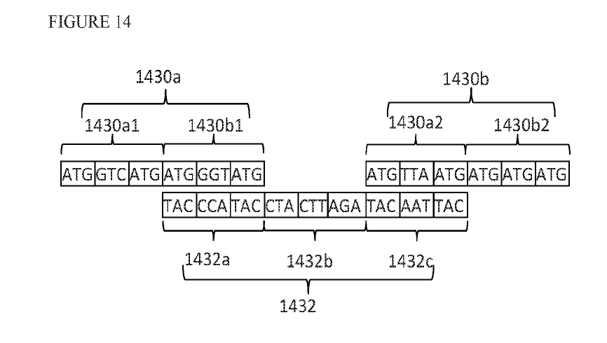

As shown in FIG. 14, every three base pairs of the DNA coded chemical may be a codon, representing a single amino acid. FIG. 14 illustrates an example of the portion of the coded chemical, DNA, which represents the path 1432 between two cities 1430a and 1430b. The coded chemical representing each city 1430a,b is divided into two portions, an arrival portion 1430a1,b1, and a departure portion 1430a2,b2. The coded chemical representing the path 1432 is divided into 3 portions, a beginning portion 1432a, and middle portion 1432b, and an end portion 1432c.

The coded chemical is designed such that the departure portion 1430b1 binds to the beginning portion 1432a of the first city 1430a and the end portion 1432c binds to the arrival portion 1430a2 of another city 1430b. The portions of the these coded chemicals bind together because the coded chemical is designed such that each of these portions are made of complementary sequences, such that they bind together through Watson-Crick base pairing. Although only a single pair of cities is shown, further connections may be made through the arrival portion 1430a1 and departure portion 1430b2 of the first and second city 1430a,b.

The middle portion 1432b of the coded chemical represents the ‘length’ (weight) of the path. When the weight of the path 1432 is high, then a DNA sequence is created which codes for amino acids which are more basic (have a high pKa). Once coded chemicals are designed which correspond to each of the cities 1430a,b and paths 1432 between them are designed, them may be inserted into a logical cognitive cell 202c. Through transcription and translation, the information in the DNA coded chemical is then translated to the equivalent information in an amino acid coded chemical. This set of amino acid coded chemicals represents the total number of possible routes through the system. The produced amino acids may then be extracted from the cell, purified, and the weighting (pKa) of the proteins tested (gel electrophoresis) to determine the optimal route through the system. The sequence of the optimal route is then determined by taking the optimal route out of the gel and using MALDI-TOF to determine the sequence of amino acid coding chemicals, which correspond to the cities and paths of the route.

In operation, DNA used as a data storage molecule containing the entire information of an organism may be copied into the next generation of the species. DNA also provides a huge storage capacity because DNA encodes data applying 4 sub units including A, G, C, and T, while current computers apply binary (0, 1) for data storage and processing.

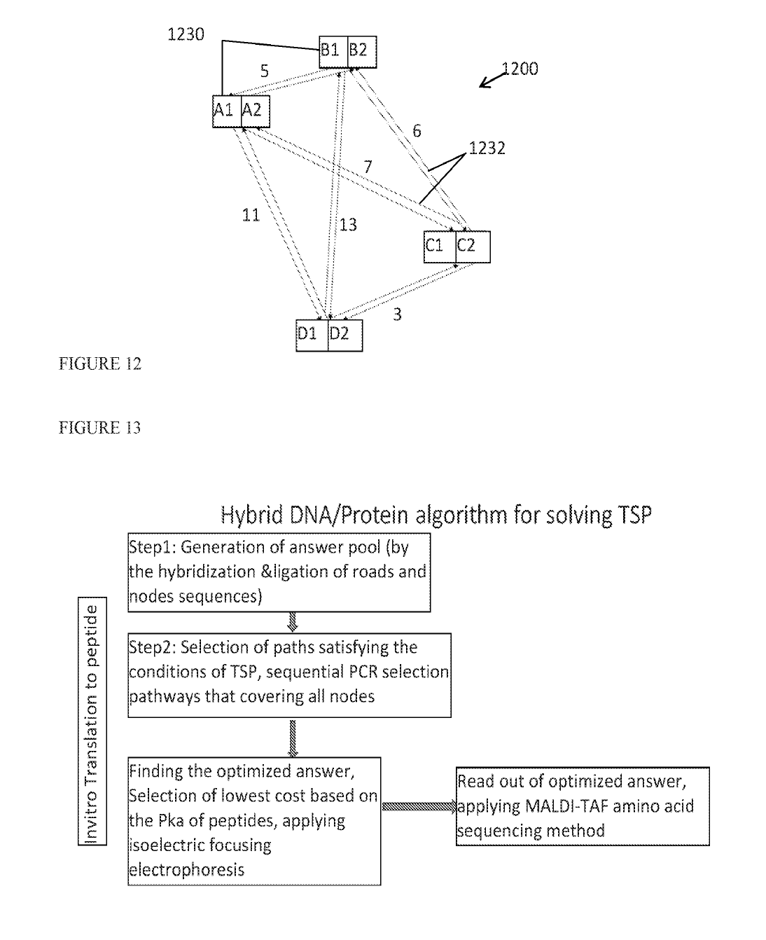

Applying the multilayer data processing and operation model system of hybrid DNA/protein codon system, the optimal solution for TSP problem created and at the final stage the optimal answer of the problem is extracted based on the amino acids' codon as shown in the Hybrid DNA/Protein algorithm of FIG. 13. FIG. 13 is a schematic diagram representing the Hybrid DNA/Protein algorithm having a bilayer coding system. This algorithm starts with a generation of an answer pool by the hybridization & ligation of roads and node sequence, then selection of paths satisfying the conditions of TSP, sequential PCR selection pathways that cover all nodes, finding the optimized answer, selection of lowest cost based on the Pka of peptides, applying iselectric focusing electrophoresis, and providing a readout of the optimized answer by applying MALDI-TAF amino acid sequenced.

The DNA sequences can be defined to correspond to each city (node) and road (edge) are referred to herein as TSP genes. The characteristics of each city, road, price, and/or other factors may be identified in the gene. TSP genes carry the information for the sequential data processing and operations. Two clusters of genes are defined, the first being the genes encoding the nucleotide information for nodes, and the second being nucleotide sequences for edges which are defined as amino acid/protein coding systems as shown in Tables 8-10 below:

TABLE 8

| | | | | | | | |
|---|---|---|---|---|---|---|---|
| A | AA.code | Met | Ala | Met | Met | Gly | Met |
| | mRNA code | AUG | GCU | AUG | AUG | GGU | AUG |
| | DNA code | ATG | GCT | ATG | ATG | GGT | ATG |
| B | AA.code | Met | Leu | Met | Met | Met | Met |
| | mRNA code | AUG | UUA | AUG | AUG | AUG | AUG |
| | DNA code | ATG | TTA | ATG | ATG | ATG | ATG |
| C | AA.code | Met | IsoLeu | Met | Met | Proline | Met |
| | mRNA code | AUG | AUU | AUG | AUG | CCU | AUG |
| | DNA code | ATG | ATT | ATG | ATG | CCT | ATG |
| D | AA.code | Met | Phenyl | Met | Met | Valine | Met |
| | mRNA code | AUG | UUU | AUG | AUG | GUU | AUG |
| | DNA code | ATG | TTT | ATG | ATG | GTT | ATG |

TABLE 9

| | | | | | | | | | |
|---|---|---|---|---|---|---|---|---|---|
| Route | Met | Gly | Met | ASP | GLU | Ser | Met | Leu | Met |
| AB | AUG | GGU | AUG | GAU | GAA | UCU | AUG | UUA | AUG |
| | TAC | CCA | TAC | CTA | CTT | AGA | TAC | AAT | TAC |
| BA | Met | Met | Met | Ser | Glu | ASP | Met | Ala | Met |
| | AUG | AUG | AUG | UCU | GAA | GAU | AUG | GCU | AUG |
| | TAC | TAC | TAC | AGA | CTT | CTA | TAC | CGA | TAC |
| BC | Met | Met | Met | GLU | Trp | His | Met | Iso | Met |
| | AUG | AUG | AUG | GAA | GCU | CAU | AUG | AUU | AUG |
| | TAC | TAC | TAC | CTT | CGA | GTA | TAC | TAA | TAC |
| CB | Met | Proline | Met | His | Trp | Glu | Met | Leu | Met |
| | AUG | CCU | AUG | CAU | GCU | GAA | AUG | UUA | AUG |
| | TAC | GGA | TAC | GTA | CGA | CTT | TAC | AAT | TAC |
| CD | Met | Proline | Met | ASP | ASP | ASP | Met | Phen | Met |
| | AUG | CCU | AUG | GAU | GAU | GAU | AUG | UUU | AUG |
| | TAC | GGA | TAC | CTA | CTA | CTA | TAC | AAA | TAC |
| DC | Met | Valine | Met | ASP | ASP | ASP | Met | IsoLeu | Met |
| | AUG | GUU | AUG | GAU | GAU | GAU | AUG | AUU | AUG |
| | TAC | CAA | TAC | CTA | CTA | CTA | TAC | TAA | TAC |
| DA | Met | Valine | Met | Lys | Cys | Lys | Met | Ala | Met |
| | AUG | GCU | AUG | UAU | AAA | UGU | AUG | GCU | AUG |
| | TAC | CAA | TAC | ACA | TTT | ATA | TAC | CGA | TAC |
| AD | Met | Gly | Met | Lys | Cys | Lys | Met | Phenyl | Met |
| | AUG | GGU | AUG | UGU | AAA | UGU | AUG | UUU | AUG |
| | TAC | CCA | TAC | ATA | TTT | ACA | TAC | AAA | TAC |
| BD | Met | Met | Met | Lys | Arg | Arg | Met | Phen | Met |
| | AUG | AUG | AUG | AAA | CGU | CGU | AUG | UUU | AUG |
| | TAC | TAC | TAC | TTT | GCA | GCA | TAC | AAA | TAC |
| DB | Met | Val | Met | Arg | Arg | Lys | Met | Leu | Met |
| | AUG | GUU | AUG | CGU | CGY | AAA | AUG | UUA | AUG |
| | TAC | CAA | TAC | GCA | GCA | TTT | TAC | AAT | TAC |

Every gene encodes the nucleotide information for synthesis of a unique peptide or protein. Since every edge in TSP has a specific weight, the unique genetic sequence of each edge is defined based on their weight (or cost of routes). The weight of each edge is defined by the coding and biochemical properties of corresponding amino acid sequences which have been defined as the second layer of coding (see, FIG. 14 and Table 8). The higher the cost of the edge (road), the more percentage of amino acid coding sequences with higher pKa (see, FIGS. 9 and 11, and Table 8).

Table 11 depicts a schematic representative of the coding sequences of each edge between different cities.

TABLE 10

| | U | | C | | A | | G | | |
|---|---|---|---|---|---|---|---|---|---|
| U | UUU | Phe/f | UCU | Ser/s | UAU | Tyr/Y | UGU | Cys/C | U |
| | UUC | | UCC | | UAC | | UGC | | C |
| | UUA | Leu/L | UCA | | UAA | STOP | UGA | STOP | A |
| | UUG | | UCG | | UAG | STOP | UGG | Trp/W | G |
| C | CUU | Leu/L | CCU | Pro/P | CAU | His/H | CGU | Arg/R | U |
| | CUC | | CCC | | CAC | | CGC | | C |
| | CUA | | CCA | | CAA | Gln/Q | CGA | | A |
| | CUG | | CCG | | CAG | | CGG | | G |
| A | AUU | lle/l | ACU | Thr/T | AAU | Asn/N | AGU | Ser/S | U |
| | AUC | | ACC | | AAC | | AGC | | C |
| | AUA | | ACA | | AAA | Lys/K | AGA | Arg/R | A |
| | AUG | Met/M | ACG | | AAG | | AGG | | G |
| G | GUU | Val/V | GCU | Ala/A | GAU | Asp/D | GGU | Gly/G | U |
| | GUC | | GCC | | GAC | | GGC | | C |
| | GUA | | GCA | | GAA | Glu/E | GGA | | A |
| | GUG | | GCG | | GAG | | GGG | | G |

Left—1st position, top—2nd position, and right—3rd position

The coding sequences of edge are made by three parts including initial (A), middle (B) and ending (C) sequences, the initial is designed as the complementary of the ending sequence of the original city (A), the middle part is designed based on the defined weight for each road, the higher weight, the higher frequency of basic amino acids (higher Pka), the ending part is designed as the complementary sequence of the target city (B) (Tables 8-10).

The DNA sequences corresponding to the nodes (or cities) may be designed with equal cost which is coded by nucleotide sequences for nonpolar amino acids. In the example of FIG. 12, the coding sequences of each city (A, B, C, D) may have parts representing the initial 9 nucleotides for the start of each city, the 9 end nucleotides of the end of each city. The sequences of cities may be on the anti-sense DNA strand, therefore the sequences of cities may be the complementary of the defined sequences in this picture.

Since in TSP the weight of each road is the main factor in the analytical process, the DNA sense strand were designed for coding the road genes (including the cost sequence). Table 10 depicts an example schematic explaining the concept of designing coding sequence of cities and roads based on hybridization mechanism. The schematic indicates an example of coding sequences for the road from city A to city B. Nucleotide hybridization and self-assembling properties of DNA molecule have been applied for parallel formation of all possible TSP solutions. For this purpose, DNA sequences related to each city divided into parts of 1, 2 and the DNA sequences related to each road divided into 3 compartments. The middle part of each road was coded based on the weight of each road. The complementary sequences of the initial and target cities relevant to each road were added to the primary weighted sequences of each road (Table 9).

After merging the sequences relevant to different cities and roads, based on the hybridization capacity of nucleotides, complementary DNA base pairs are hybridizing together all possible pathways between different cities will be formed (Table 9).

The present hybrid DNA/protein algorithm includes two layers of operations. Firstly, synthesizing a solution pool and secondly extraction of optimal solution or shortest path from the solution pool. For the first layer, biochemical operations are directly employed on genetic networks by merging the DNA sequences corresponding all edges and nodes together to create a solution pool containing all possible combinations of roads and nodes. For the second part through the translation operation, all nucleotide sequences converted to their relevant amino acids' codons as the second layer of coding and data processing. Finally, a peptide operation approach is employed to select the optimal solution or shortest path for the TSP (Table 9).

Before going through the Hybrid DNA/protein algorithm, two different sets of biochemical operations are explained here, including the synthesis of answer pool by DNA-based operations and then the extraction of optimal solution from the answer pool applying protein-based operations.

The cognitive system uses biochemical operations in a hybrid DNA/Protein algorithm for solving TSP: Synthesis of DNA genes of TSP is based on standard chemical oligonucleotide synthesis. Gene sequences of cities and costs are designed by 18 and 27 per single stranded DNA (ssDNA) respectively (Table 10). Gene sequences of roads and cities ae designed in a way for assembly to provide the possible combinations of cities and roads (costs) based on the hybridization reaction between the overlapping sequences in cities and roads (Table 10). Each road gene has been made of 3 parts which contains the cost information in the middle (Part I) and two linker parts (Part II and III) on the right and left side of cost information part. Linker parts are connecting the cost part of each road to the original and goal city gene sequences through the hybridization reaction. Linker part A is the complement of the second part (pink color) of the original city and the linker part C is the complement of the first part (yellow color) of the goal city (FIG. 6). All possible paths including different combinations of cities and roads are generated by the hybridization reaction between the sequences of cities and their complements flanking parts on both sides of cost sequences.

Merge operation of cities and costs in the hybrid DNA/protein algorithm is based on the sequential hybridization and ligation reactions between the Watson-Crick complimentary DNA sequences. Briefly, all gene sequences of cities and costs are merged together for the hybridization reaction (including denaturation of DNA sequences at 95° C., following with annealing/hybridization reaction at 20° C.). Sequentially, merge operation is completed by providing the appropriate condition for ligation reaction (including 4 μl Ligase reaction buffer, 30 fmole of vector DNA, 90 fmole insert DNA, up to 15 μl of DNAse/RNAse free H₂O, 1 μl of T4 DNA Ligase with the total reaction volume 20 μl, 5 min incubation time) applying Thermo Fisher Scientific T4 ligation kit (Termo Fisher Scientific, Cat No. 15224-017).

Pathways satisfying the requirements of TSP may be solved through solid phase hybridization/ligation reactions, gel electrophoresis, affinity separation and PCR. The satisfying requirements of TSP are achieved by applying sequential biologically inspired operations including solid phase hybridization/ligation reaction, gel electrophoresis, affinity separation and PCR. In order to generate all possible paths starting and ending with a specific city (e.g., city A), Initially the 5′ end of city A gene sequence is conjugated to a magnetic bead for the next step selection by attachment to a solid phase magnetic column. Then gene sequences of all cities exception of city A and all road sequences are added into the reaction solution. The gene sequences of all cities and roads annealed together through the Watson-Crick hybridization reaction as explained above. After the completion of hybridization/ligation reaction, the remaining city and road gene sequences that did not involve in to the reaction, are removed from the reaction medium by washing.

The merge operation of TSP may be completed by adding the gene sequence of city A at the final stage. Paths that satisfying the TSP requirement of covering 4 cities and roads are defined through their length by gel electrophoresis operation. Paths that are visiting each city once were defined by affinity separation operation through several affinity columns, containing the complementary sequences of each city. In order to provide enough template DNA satisfying the TSP requirements, for the next layer of coding which is protein translation, all paths initiating and ending with city A are amplified by PCR operation (applying specific forward primer for the first part of city A and back ward primer for the second part of city A). PCR is defined based on the QIAGEN multiplex PCR kit instruction (QIAGEN, Cat No. 206143), for 34 cycles, 30 S at 95° C., 30 S at Tm-5 C, 30 S at 72° C. and initial denaturation for 15 C. Briefly, 2 μl of 2× QIAGEN Multiplex PCR master mix, 5 μl of 10× primer mix (2 μM of each primer), 1 μg of template DNA and RNA free water with the final volume of 50 μl.

Protein translation, extraction, analysis operations include transferring of oligonucleotides' answer pool into expression vectors, in vitro translation assays, protein extraction and analysis assays were performed respectively, in order to extract the optimal solution for a defined TSP problem based on the primary definition of DNA sequences relevant to each city and road.

To this end, isoelectric-focusing and 2D-electrophoresis are defined as the final operations for finding the optimal solution based on the weight and pKa of synthesized peptides. Matrix-Assisted Laser Desorption/Ionization on a reflection Time-Of-Flight (MALDI-TOF) (which is a standard amino acid sequencing method) is defined as the read-out operation of the optimal solution based on the amino acid sequences.

Therefore, in the first layer of algorithm through the self-hybridization and self-assembling properties of DNA base pairs, a TSP answer pool is generated. Then, in the following stages the optimal solution (the shortest and most economical pathway) are extracted by translation of synthetized DNA sequences in to the next layer of coding which is defined as protein translation layer. Finally, based on the coding properties of amino acids the optimal solution is extracted from the peptide answer pool according to the solution of Table 8.

The hybrid DNA/Peptide algorithm of FIG. 13 may be used to solve the TSP by: 1) inputting a complete set of all DNA sequences defined as the gene sequences of each city and road in a TSP merge operation, 2) inputting the output of first layer (DNA operation layer) and representing TSP satisfied conditions which is a tour passing all nodes exactly once, 3) integrating TSP satisfied conditions into the protein translation operation by providing of the translation required elements for an in vitro protein translation operation, 4) separating the TSP optimal solution or the most economical path which is represented by a peptide with the lowest pKa value from other candidate paths and applying isoelectric focusing and 2 dimensional electrophoresis operations, and 5) providing the TSP optimal solution read-out by amino acid sequencing of the peptide fraction with the lowest pKa value through MALDI-TOF protein sequencing operation.

The above description is for the purpose of teaching the person of ordinary skill in the art how to practice the aspects of the present application, and it is not intended to detail all those obvious modifications and variations of it which will become apparent to the skilled worker upon reading the description. It is intended, however, that all such obvious modifications and variations be included within the scope of the present application. The aspects and embodiments are intended to cover the components and steps in any sequence which is effective to meet the objectives there intended, unless the context specifically indicates the contrary.

The cognitive system may be used in a variety of applications, such as for optimizing the efficiency of a multi-node process comprising applying a hybrid DNA/protein algorithm to a nondeterministic polynomial problem (NP) comprising multiple nodes, wherein the NP (e.g., TSP problem) represents the process. The process may involve a logistics process, procurement, package delivery process, wiring of a computer or circuit board, data array clustering, scheduling of multiple tasks with no intermediate storage or down-time, vehicle routing (autonomous, self-driving car or truck, drone aircraft, etc.). The routing may be for package delivery, surveillance, etc. The process may also involve a method of optimizing the efficiency a vehicle routing process for an autonomous vehicle, comprising: applying a hybrid DNA/protein algorithm to a nondeterministic polynomial problem (NP) comprising multiple nodes, wherein each of said multiple nodes represents a point within the vehicle routing process and each point only occurs once, thereby establishing a optimally efficient route for said autonomous vehicle, and programming said optimally efficient route into said autonomous vehicle.

The multilayer biologically inspired coding may include double, triple or multiple layers of biologically inspired coding systems including DNA (genetic coding), epigenetic (Histone acetylation, DNA methylation), Zinc finger's/TALEN's specific DNA recognition coding, ON/OFF promoter inducible systems (applying inducible switches such as TET, IPTG, Light, Steroids) induction systems, mRNA alternative splicing coding, linc RNA (Long intergenic noncoding RNAs), guide RNA/CRISPR/CAS-9 codon integration and targeted genome edition system, genetic integration and recombination coding systems, amino acid coding system, enzymatic and protein signal transduction systems, hormonal and neurotransmitters systemic signal transmission and coding systems, electric signal transduction systems through membrane ion channels and receptors as well as intracellular organelles, chemical and electronic signal transductions between different cell types such as cardiac pacemaker cells and neural cells locally or systematic.

The cognitive system may be used for solving NP problems relating to various technical fields (e.g., engineering), technologies (e.g., airlines, planes, ships, trains, trucks, taxis, ride-share services or cars), industries (e.g., shipping or freight services), cryptographic and/or scheduling applications, public space security problems (e.g., efficient number of security guards, hospital spacing), treatment of diseases, pharmaceuticals (e.g., for optimization of recombinant proteins), and/or chemicals (e.g., catalyst functionality of enzymes), industrial scale use, language and string processing, game and puzzle design and layout, drilling of printed circuit boards, mask plotting in printed circuit board production, overhauling gas turbine engines, X-ray crystallography, computer wiring, order-picking of goods, vehicle routing, printing press scheduling, crew scheduling, interview scheduling, hot rolling scheduling, military mission planning, and design of global navigation satellite system surveying networks, wiring of a computer or circuit board, data array clustering, sand scheduling of multiple tasks with no intermediate storage or down-time, data analysis (e.g., high throughput biological data), data storage, etc.

While the embodiments are described with reference to various implementations and exploitations, it will be understood that these embodiments are illustrative and that the scope of the inventive subject matter is not limited to them. Many variations, modifications, additions and improvements are possible. For example, various combinations of one or more of the features provided herein may be used.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

Insofar as the description above and the accompanying drawings disclose any additional subject matter that is not within the scope of the claim(s) herein, the inventions are not dedicated to the public and the right to file one or more applications to claim such additional invention is reserved. Although a very narrow claim may be presented herein, it should be recognized the scope of this invention is much broader than presented by the claim(s). Broader claims may be submitted in an application claims the benefit of priority from this application.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

What is claimed is:

1. A synthetic cognitive cell for automatically generating an output based on an environmental input, the cognitive cell comprising:
an operator comprising chemical agents; and
a coded chemical comprising polymers, each of the polymers comprising a sequence of affinity blocks of molecular groups arranged in predetermined patterns to define a multi-layered base code, each of the affinity blocks comprises a monomer with a sidechain, the sidechains having affinities to each other, at least a portion of the affinity blocks forming a gate switch defining a bridge between the environmental inputs and the chemical agent whereby, upon exposure to the environmental inputs, the gate switches trigger the chemical agent to perform an operation,
wherein the coded chemical and at least one chemical agent is contained within a natural or synthetic membrane.

2. The synthetic cognitive cell of claim 1, wherein the polymer is selected from the group consisting of natural or synthetic deoxyribonucleic acid (DNA), plasmid, graphene, aptamer, synthetic polysaccharide, enzyme sensitive nanopolymer, peptides, proteins, synthetic nanopolymers, polyethylene glycol, silica, and combinations thereof.

3. The synthetic cognitive cell of claim 1, wherein the polymers are modified by the sidechains, the sidechains being a natural or synthetic chemical selected from the group consisting of a hydroxyl group, an amino group, a carboxyl group, a carbonyl group, phosphate, sulfide, sulfoxide, natural and synthetic antigens, and combinations thereof.

4. The synthetic cognitive cell of claim 1, wherein the polymers are modified by at least one of the sidechains, the modified polymers comprising synthetic polymers with synthetic microbeads made of nanoparticles selected from the group consisting of silica, poly-DL-lactic-co-glycolic, and combinations thereof.

5. The synthetic cognitive cell of claim 1, wherein the chemical agents are selected from the group consisting of ADP, ATP, GDP, GTP, NAD, FAD, tRNA, Ribulose 1,5 bisphosphate, cobaloxime, phosphoglycerate kinase, glyceraldehyde-3-phosphate dehydrogenase, hexoisomerase, aldolase, oxygenase, glyceraldehyde dehydrogenase, fructose 1,6 bisphosphatase, phosphoglycerate kinase, tetracycline, rttA, nucleotides, ligase, amino acids, magnesium chloride, primers, glucose, vitamins, and combinations thereof.

6. The synthetic cognitive cell of claim 1, wherein the molecular groups define storage units corresponding to the environmental inputs and the outputs, the predetermined patterns arranged to provide a selected one or more of the outputs based on a selected one or more of the environmental inputs.

7. The synthetic cognitive cell of claim 1, further comprising a buffer selected from the group consisting of water, a phosphate buffer, an amine buffer, and combinations thereof.

8. The synthetic cognitive cell of claim 1, wherein the environmental input comprises light and the output is selected from the group consisting of glucose and ATP, cellulose, chitosan, silk, insulin, and combinations thereof.

9. The synthetic cognitive cell of claim 8,
wherein the polymers comprises synthetic DNA, the polymers modified by the sidechains;
wherein the chemical agents is one selected from the group consisting of Ribulose 1,5 bisphosphate, NAD, ADP, ATP, Cobalaxime, and combinations thereof; and
wherein the gate switch is one selected from the group consisting of tetracycline, IPTG, tamoxifen, and combinations thereof.

10. The synthetic cognitive cell of claim 1, wherein the environmental input comprises exposure to an external chemical after damage, and the output comprises cell growth.

11. The synthetic cognitive cell of claim 10,
wherein the polymers comprise:
a first polymer selected from the group consisting of TRE, FGF, COL, and combinations thereof;
a second polymer selected from the group consisting of CMV, an rttA DNA gene sequence, and combinations thereof; and
a third polymer selected from the group consisting of an enzyme sensitive nanopolymer modified by a sidechain comprising a peptide;
wherein the chemical agents are selected from the group consisting of tetracycline, rttA protein, and combinations thereof; and
wherein the gate switch is one selected from the group consisting of TRE, the enzyme sensitive nanopolymer, enzyme sensitive nanopolymer, tetracycline and combinations thereof.

12. The synthetic cognitive cell of claim 1, wherein the environmental input comprises fuel and the output comprises route, and a result of the output comprises an optimal solution.

13. The synthetic cognitive cell of claim 12,
wherein the polymers are selected from the group consisting of synthetic DNA, PEG, silica and combinations thereof, the polymers modified by the sidechains;
wherein the chemical agents are selected from the group consisting of nucleotides, MgCl2, ligase, amino acids, tRNAs, ATP, primers, and combinations thereof; and
wherein the gate switches comprise an affinity between sidechains of the polymers.

14. The synthetic cognitive cell of claim 1, further comprising a catalyst selected from the group consisting of rhodium, platinum, TiO2 nanoparticles, pyrroloquinoline quinone and graphenes nanoparticles.

15. A synthetic cognitive system for automatically generating outputs, comprising:
at least one environmental input; and
synthetic cognitive cells, each synthetic cognitive cell comprising:
an operator comprising a chemical agent; and
a coded chemical comprising polymers, each of the polymers comprising a sequence of affinity blocks of molecular groups arranged in predetermined patterns to define a multi-layered base code, each of the affinity blocks comprises a monomer with a sidechain, the sidechains having affinities to each other, at least a portion of the affinity blocks forming a gate switch defining a bridge between the environmental inputs and the chemical agent whereby, upon exposure to the environmental inputs, the gate switches trigger the chemical agent to perform an operation.

16. The synthetic cognitive system of claim 15, wherein a first of the synthetic cognitive cells is responsive to the at least one environmental input and generates a first of the outputs, and wherein the first of the outputs acts as the at least one environmental input for a second of the cognitive cells for the synthetic generating a second of the outputs.

17. The synthetic cognitive system of claim 15, wherein the outputs are generated by the operation, the cognitive system further comprising a translator to convert the outputs to results, the results comprising energy, growth, a solution, and combinations thereof.

18. A method of making a synthetic cognitive cell for automatically generating an output based on an environmental input, the method comprising:
providing an operator comprising chemical agents;
providing polymers comprising monomers;
forming a coded chemical by:
selectively forming affinity blocks by modifying the monomers with sidechains, the sidechains having an affinity to each other; and
arranging a sequence of the affinity blocks of the polymer into predetermined patterns of molecular groups defining a multi-layered base code and into gate switches defining a bridge between the environmental input and the chemical agent such that, on exposure to the environmental input, chemical reactions between the chemical agent and the coded chemical perform an operation;
mixing the operator with the coded chemical to form a coded mixture; and
applying the coded mixture to a membrane.

19. The method of claim 18, wherein the arranging comprises storing data in the coded chemical by assigning each of the molecular groups to an identifier corresponding to an environmental input.

20. The method of claim 19, wherein the arranging comprises selecting the gate switches corresponding to one of the molecular groups and one of the gate switches to each of the environmental outputs.
